(19) United States
(12) Patent Application Publication
(10) Pub. No.: US 2023/0392185 A1
(43) Pub. Date: Dec. 7, 2023

(54) METHODS AND SYSTEMS FOR DETERMINING SUITABILITY OF COMPOSITIONS FOR INHIBITING GROWTH OF POLYMICROBIAL SAMPLES

(71) Applicant: CAP DIAGNOSTICS, LLC, DBA PATHNOSTICS, Irvine, CA (US)

(72) Inventors: David A. Baunoch, Irvine, CA (US); Miguel F.R. Penaranda, Irvine, CA (US); Michael L. Opel, Irvine, CA (US); Maher Badir, Irvine, CA (US); Natalie Luke, Irvine, CA (US)

(21) Appl. No.: 18/451,748

(22) Filed: Aug. 17, 2023

Related U.S. Application Data
(63) Continuation-in-part of application No. PCT/US22/16816, filed on Feb. 17, 2022.
Application No. PCT/US22/16816 is a continuation-in-part of application No. 17/178,091, filed on Feb. 17, 2021.
Application No. 17/178,091 is a continuation-in-part of application No. PCT/US21/27336, filed on Apr. 14, 2021.
Application No. PCT/US21/27336 is a continuation-in-part of application No. 17/335,767, filed on Jun. 1, 2021, now Pat. No. 11,746,371.
Application No. 17/335,767 is a continuation-in-part of application No. 17/830,227, filed on Jun. 1, 2022.
Application No. 17/830,227 is a continuation-in-part of application No. 17/178,091, filed on Feb. 17, 2021.
Application No. PCT/US21/27336 is a continuation-in-part of application No. 17/178,091, filed on Feb. 17, 2021.
Application No. 17/178,091 is a continuation-in-part of application No. 16/848,651, filed on Apr. 14, 2020, now Pat. No. 11,053,532.
Application No. 16/848,651 is a continuation-in-part of application No. 16/848,651, filed on Apr. 14, 2020, now Pat. No. 11,053,532.
Application No. 16/848,651 is a continuation-in-part of application No. 16/216,751, filed on Dec. 11, 2018, now abandoned.
Application No. 16/216,751 is a continuation of application No. 15/957,780, filed on Apr. 19, 2018, now Pat. No. 10,160,991.
(60) Provisional application No. 63/195,502, filed on Jun. 1, 2021.
(60) Provisional application No. 63/251,433, filed on Oct. 1, 2021.
(60) Provisional application No. 63/195,502, filed on Jun. 1, 2021.
(60) Provisional application No. 63/047,846, filed on Jul. 2, 2020.
(60) Provisional application No. 63/063,093, filed on Aug. 7, 2020.
(60) Provisional application No. 63/111,287, filed on Nov. 9, 2020.
(60) Provisional application No. 63/119,328, filed on Nov. 30, 2020.
(60) Provisional application No. 62/977,637, filed on Feb. 17, 2020.
(60) Provisional application No. 62/978,149, filed on Feb. 18, 2020.
(60) Provisional application No. 62/988,186, filed on Mar. 11, 2020.
(60) Provisional application No. 63/009,337, filed on Apr. 13, 2020.
(60) Provisional application No. 63/047,846, filed on Jul. 2, 2020.
(60) Provisional application No. 63/063,093, filed on Aug. 7, 2020.
(60) Provisional application No. 63/111,287, filed on Nov. 9, 2020.
(60) Provisional application No. 63/119,328, filed on Nov. 30, 2020.
(60) Provisional application No. 62/924,614, filed on Oct. 22, 2019.
(60) Provisional application No. 62/928,815, filed on Oct. 31, 2019.
(60) Provisional application No. 62/956,923, filed on Jan. 3, 2020.
(60) Provisional application No. 62/977,637, filed on Feb. 17, 2020.
(60) Provisional application No. 62/978,149, filed on Feb. 18, 2020.
(60) Provisional application No. 62/988,186, filed on Mar. 11, 2020.
(60) Provisional application No. 63/009,337, filed on Apr. 13, 2020.
(60) Provisional application No. 62/487,395, filed on Apr. 19, 2017.

(51) Int. Cl. C12Q 1/08; G01N 21/59

(52) U.S. Cl. CPC C12Q 1/08; G01N 21/59; G01N 2021/593; G01N 21/5907

(57) ABSTRACT

Methods for identifying and providing information about inhibiting growth of polymicrobial infections, including but not limited to providing statistics or information about the likelihood of success in inhibiting growth of a polymicrobial infection with particular compositions or therapeutic solutions. The methods herein feature detection and identification of organisms of the polymicrobial sample (e.g., polymicrobial infection), phenotypic pooled sensitivity tests for determining the susceptibility or resistance of the polymicrobial sample (e.g., polymicrobial infection) in the sample to an antibiotic or other therapeutic agent, and identification of resistance genes, e.g., genetic markers that may indicate resistance to a particular treatment. Together, the data can be applied against databases of antibiotic/therapeutic susceptibility or resistance for particular known polymicrobial samples (e.g., polymicrobial infections) in order to provide information related to the likelihood of success of one or more therapeutic solutions for the polymicrobial sample (e.g., polymicrobial infection).

### CROSS-REFERENCES TO RELATED APPLICATIONS

This application is a continuation-in-part and claims benefit of PCT/US22/16816 filed Feb. 17, 2022, which claims benefit of U.S. patent application Ser. No. 17/178,091 filed Feb. 17, 2021, PCT Application No. PCT/US21/27336 filed Apr. 14, 2021, U.S. patent application Ser. No. 17/335,767 filed Jun. 1, 2021, U.S. Provisional Application No. 63/195,502 filed Jun. 1, 2021, and U.S. Provisional Application No. 63/251,433 filed Oct. 1, 2021, the specifications of which are incorporated herein in their entirety by reference.

This application is also continuation-in-part and claims benefit of U.S. patent application Ser. No. 17/830,227, filed Jun. 1, 2022, which is a non-provisional and claims benefit of U.S. Provisional Application No. 63/195,502 filed Jun. 1, 2021, the specification of which is incorporated herein in its entirety by reference.

U.S. patent application Ser. No. 17/830,227, filed Jun. 1, 2022, is also a continuation-in-part and claims benefit of U.S. patent application Ser. No. 17/178,091 filed Feb. 17, 2021.

PCT/US21/27336 claims benefit of U.S. patent application Ser. No. 17/178,091 filed Feb. 17, 2021 and U.S. patent application Ser. No. 16/848,651 filed Apr. 14, 2020, now U.S. Pat. No. 11,053,532, the specifications of which are incorporated herein in their entirety by reference.

PCT/US21/27336 also claims benefit of U.S. Patent Application No. 63/047,846 filed Jul. 2, 2020, U.S. Patent Application No. 63/063,093 filed Aug. 7, 2020, U.S. Patent Application No. 63/111,287 filed Nov. 9, 2020, and U.S. Patent Application No. 63/119,328 filed Nov. 30, 2020, the specifications of which are incorporated herein in their entirety by reference.

U.S. patent application Ser. No. 17/178,091 is a non-provisional and claims benefit of U.S. Provisional Patent Application No. 62/977,637 filed Feb. 17, 2020, U.S. Provisional Patent Application No. 62/978,149 filed Feb. 18, 2020, U.S. Provisional Patent Application No. 62/988,186 filed Mar. 11, 2020, U.S. Provisional Patent Application No. 63/009,337 filed Apr. 13, 2020, U.S. Provisional Patent Application No. 63/047,846 filed Jul. 2, 2020, U.S. Provisional Patent Application No. 63/063,093 filed Aug. 7, 2020, U.S. Provisional Patent Application No. 63/111,287 filed Nov. 9, 2020, and U.S. Provisional Patent Application No. 63/119,328 filed Nov. 30, 2020, the specifications of which are incorporated herein in their entirety by reference.

U.S. patent application Ser. No. 17/178,091 is also a continuation-in-part and claims benefit of U.S. patent application Ser. No. 16/848,651 filed Apr. 14, 2020, now U.S. Pat. No. 11,053,532.

U.S. patent application Ser. No. 17/335,767 is a Divisional and claims benefit of U.S. patent application Ser. No. 16/848,651 filed Apr. 14, 2020, now U.S. Pat. No. 11,053,532.

U.S. patent application Ser. No. 16/848,651 is a non-provisional and claims benefit of U.S. Patent Application No. 62/924,614 filed Oct. 22, 2019, U.S. Patent Application No. 62/928,815 filed Oct. 31, 2019, U.S. Patent Application No. 62/956,923 filed Jan. 3, 2020, U.S. Patent Application No. 62/977,637 filed Feb. 17, 2020, U.S. Patent Application No. 62/978,149 filed Feb. 18, 2020, U.S. Patent Application No. 62/988,186 filed Mar. 11, 2020, and U.S. Patent Application No. 63/009,337 filed Apr. 13, 2020, the specifications of which are incorporated herein in their entirety by reference.

U.S. patent application Ser. No. 16/848,651 is also a continuation-in-part and claims benefit of U.S. patent application Ser. No. 16/216,751 filed Dec. 11, 2018, now abandoned, which is a continuation and claims benefit of U.S. patent application Ser. No. 15/957,780 filed Apr. 19, 2018, now U.S. Patent No. which is a non-provisional and claims benefit of U.S. Patent Application No. 62/487,395, filed Apr. 19, 2017, the specifications of which are incorporated herein in their entirety by reference.

### BACKGROUND OF THE INVENTION

### Field of the Invention

The present application is related to methods and systems for identifying polymicrobial samples, e.g., polymicrobial infections, as well as methods of determining suitability of one or more compositions for inhibiting growth of the polymicrobial sample, e.g., providing information related to the likelihood of success of inhibiting growth of the polymicrobial sample with the one or more compositions. The present application also describes methods for providing information to a user regarding the polymicrobial sample, such as but not limited to information regarding the suitability of the compositions for inhibiting growth of a polymicrobial sample.

### Background Art

Infectious diseases can affect multiple organ systems and are responsible for significant morbidity, mortality, and economic impact. Infectious agents most often present as complex polymicrobial infections rather than as a single pathogen infection. Within the body, these polymicrobial infections cooperate with each other through various interactions changing both the type of antibiotics the organisms are susceptible to but also the level of antibiotics required to treat the infection as well as the virulence of the individual pathogens.

The present application describes methods for determining suitability of one or more compositions for inhibiting growth of the polymicrobial sample, e.g., providing information related to the likelihood of success of inhibiting growth of the polymicrobial sample with the one or more compositions. The present application also describes methods of providing information related to the suitability of one or more compositions for inhibiting growth of the polymicrobial sample.

The present invention describes culture-independent approaches to detecting and identifying bacteria, such as a multiplex PCR-based method (DNA-based) (M-PCR). For example, M-PCR, does not require growth and targets uropathogenic species. M-PCR provides semi quantitative information that is comparable to culture and is presented in ranges such as <9,999 cells/mL, 10,000-49,999 cells/mL, 50,000-99,999 cells/mL, and ≥100,000 cells/mL. Once again, the number of cells/mL is comparable to CFU's. The vast majority of uropathogens identified by M-PCR are found at concentrations ≥100,000 cells/mL demonstrating that detection is not due to the increased sensitivity with respect to PCR; instead is due to the inability of SUC to detect the bacteria. The concentration of the non-culturable organisms is on par with those that are culturable. In a large prospective trial of symptomatic patients, these uropathogens detected by M-PCR were found to be linked to symptomatology.

The present invention also describes Pooled Antibiotic Susceptibility Testing (P-AST), (U.S. Pat. No. 10,160,991, the specification of which is incorporated herein in its entirety by reference), which involves simultaneously growing all detected bacteria together in the presence of antibiotics and then measuring susceptibility. Thus, P-AST considers interactions between cohabiting bacterial species and may serve as a more accurate predictor of antibiotic susceptibility.

The present invention also describes genotypic antibiotic resistance (ABR) testing, wherein the bacteria of the polymicrobial infection are tested for particular genetic markers. The odds of resistance of the polymicrobial infection to particular antibiotics are applied to determine appropriate therapeutic solutions.

The present invention describes the use of said methods and systems for identifying polymicrobial infections in urine, identifying or providing therapeutic solutions for treating said polymicrobial infections in the urine. For example, the present invention provides methods and systems for allowing the rapid identification of UTIs, and the rapid identification of a treatment solution for the UTIs. The present invention is not limited to polymicrobial infections associated with urine.

### BRIEF SUMMARY OF THE INVENTION

Disclosed herein are methods of determining suitability of one or more compositions for inhibiting growth of the polymicrobial sample, e.g., providing information related to the likelihood of success of inhibiting growth of the polymicrobial sample with the one or more compositions. The present application also describes methods for providing information to a user regarding the polymicrobial sample, such as but not limited to information regarding the suitability of the compositions for inhibiting growth of a polymicrobial sample (e.g., statistics or information about the likelihood of success in inhibiting growth of a polymicrobial infection with particular compositions or therapeutic solutions, etc.). The present invention also describes the documents, presentation, or other media for providing said information to the user.

The present invention also features methods for detection and identification of organisms of the polymicrobial sample (e.g., polymicrobial infection), phenotypic pooled sensitivity tests for determining the susceptibility or resistance of the polymicrobial sample (e.g., polymicrobial infection) in the sample to an antibiotic or other therapeutic agent, and identification of resistance genes, e.g., genetic markers that may indicate resistance to a particular treatment. Together, the data can be applied against databases of antibiotic/therapeutic susceptibility or resistance for particular known polymicrobial samples (e.g., polymicrobial infections) in order to provide information related to the likelihood of success of one or more therapeutic solutions for the polymicrobial sample (e.g., polymicrobial infection). The present invention also features methods and systems for identifying polymicrobial infections and identifying or providing therapeutic solutions for polymicrobial infections. The present invention also features methods and systems for treating polymicrobial infections.

The methods herein feature: (1) detection and identification of organisms (e.g., bacteria or other infectious agents) of the polymicrobial infection, (2) pooled antibiotic susceptibility tests for determining the susceptibility or resistance of the polymicrobial infection in the sample to an antibiotic or other therapeutic agent, and (3) identification of resistance genes in the infectious agents in the polymicrobial infection, e.g., genetic markers that may indicate resistance to a particular antibiotic or other therapeutic agent or treatment. Together, the data from (1), (2), and (3) can be applied (e.g., using databases of antibiotic/therapeutic susceptibility or resistance for particular known polymicrobial infections) in order to provide one or more therapeutic solutions for the polymicrobial infection.

In some embodiments, the organisms are bacteria. The present invention is not limited to bacterial infectious agents and may include viruses, fungi, protozoa, bacteria, or a combination thereof.

The present invention describes a method for providing a therapeutic solution to treat polymicrobial infection or suspected polymicrobial infection in a patient. In some embodiments the method comprises subjecting a portion of a sample obtained from a source of the polymicrobial infection in the patient to genetic identification testing to detect and identify one or more organisms in the sample. The method may further comprise subjecting a portion of the sample to genetic resistance marker testing to detect and identify one or more resistance genes in the organisms identified (e.g., resistance genes that confer resistance to one or more therapeutic agents). The method may further comprise subjecting a portion of the sample to pooled phenotypic antibiotic resistance testing (pooled susceptibility testing), wherein phenotypic antibiotic resistance testing either or both: identifies one or more therapeutic agents to which the polymicrobial infection is resistant, and/or identifies one or more therapeutic agents to which the polymicrobial infection is susceptible. The one or more organisms of the polymicrobial infection in the sample are not first isolated before phenotypic antibiotic resistance testing. The method may further comprise applying the results from genetic identification testing, genetic resistance marker testing, and pooled antibiotic susceptibility testing to a database, e.g., predetermined thresholds of a database. The analysis identifies one or more therapeutic agents that are effective for treating the polymicrobial infection (e.g., a “therapeutic solution”).

The present invention also features a method for treating a polymicrobial infection or suspected polymicrobial infection in a patient in need thereof. In some embodiments, the method comprises subjecting a portion of a sample obtained from a source of the polymicrobial infection in the patient to genetic identification testing to detect and identify one or more organisms in the sample. The method may further comprise subjecting a portion of the sample to genetic resistance marker testing to detect and identify one or more resistance genes in the organisms identified (e.g., resistance genes that confer resistance to one or more therapeutic agents). The method may further comprise subjecting a portion of the sample to pooled phenotypic antibiotic resistance testing, wherein phenotypic antibiotic resistance testing either or both: identifies one or more therapeutic agents to which the polymicrobial infection is resistant, and/or identifies one or more therapeutic agents to which the polymicrobial infection is susceptible. The one or more organisms of the polymicrobial infection in the sample are not first isolated before phenotypic antibiotic resistance testing. The method may further comprise applying the results from genetic identification testing, genetic resistance marker testing, and pooled antibiotic susceptibility testing to a database, e.g., predetermined thresholds of a database. The analysis identifies one or more therapeutic agents that are effective for treating the polymicrobial infection (e.g., a “therapeutic solution”). The method may further comprise administering at least one therapeutic agent identified to the patient, wherein the at least one therapeutic agent is effective for treating the polymicrobial infection.

The database that indicates which therapeutic agents are effective for treating a number of different polymicrobial infections may be generated by a compilation of results of phenotypic antibiotic resistance testing, genetic resistance marker testing for a plurality of different polymicrobial infections.

In certain embodiments, the therapeutic solution may comprise one applicable therapeutic agent. In some embodiments, the therapeutic solution comprises two or more applicable therapeutic agents. In some embodiments, the therapeutic solution comprises three or more applicable therapeutic agents. In some embodiments, the therapeutic solution comprises four or more applicable therapeutic agents. In some embodiments, the therapeutic solution comprises five or more applicable therapeutic agents.

With reference to any of the embodiments herein, the method may further comprise compiling a data set that includes one or more data points selected from: (i) results of phenotypic antibiotic resistance testing, (ii) results of genetic identification testing, (iii) results of genetic resistance marker testing, (iv) therapeutic agents to which the polymicrobial infection is expected to have increased resistance, (v) therapeutic agents to which the polymicrobial infection is expected to have decreased resistance, (vi) suggested therapeutic agents, and (vii) formulation of suggested therapeutic agents.

In certain embodiments, the method further comprises generating a report that communicates the data set. In certain embodiments, the report features a chart and/or a table and/or a diagram. In certain embodiments, the method further comprises providing the report to a medical professional, wherein the report communicates recommendations for treatment for the patient.

In certain embodiments, the method further comprises measuring a number or concentration of organisms present in the sample. In some embodiments, the method further comprises Extended-Spectrum Beta-lactamase (ESBL) testing. In some embodiments, the method further comprises testing for genes associated with Clostridium difficile. In some embodiments, the method further comprises determining a microbial inhibitory concentration (MIC) for organisms of the polymicrobial infection.

In certain embodiments, the genetic identification testing comprises PCR, fluorescence in situ hybridization (FISH), culture, mass spectrometry, electrochemical biosensing, automated biochemical identification, flow cytometry, or a combination thereof. In certain embodiments, the genetic resistance marker testing comprises PCR or sequencing. In certain embodiments, the pooled phenotypic antibiotic resistance testing comprises introducing fractions of the portion of the sample to one or more media samples, each media sample comprising a therapeutic agent, incubating (e.g., under conditions suitable for growth) the media samples with the fractions, and subsequently measuring viability of organisms in the media samples after incubation. In certain embodiments, the media samples are in test tubes, wells of a culture plate, an agar plate, or a microscope slide. In certain embodiments, the viability of the organisms is measured by optical density (OD), fluorescence, or chemiluminescence.

In certain embodiments, the sample has at least one resistance marker. In certain embodiments, the sample has at least 2 resistance markers. In certain embodiments, the sample has at least 3 resistance markers. In certain embodiments, the at least one resistance marker is a mecA gene, a vanA/B gene, a TEM gene, a SHV gene, a OXA gene, a CTX-M gene, a KPC gene, a NDM gene, an OXA gene, a VIM gene, an IMP gene, or a combination thereof. In certain embodiments, the one or more resistance genes is ErmA+Erm B, TEM, CTX-M group 1, SHV, VEB, OXA-1, CTX-M group 2, CTX-M group 9, CTX-M group 8/25, PER-1, PER-2, GES, blaNDM-1, VIM, KPC, IMP-2 group, IMP-1 group, OXA-23, IMP-16, IMP-7, OXA-72, OXA-40, OXA-58, OXA-48, NDM, blaOXA-48, QnrA, QnrB, mecA, ampC, FOX, ACC, DHA, MOX/CMY, BIL/LAT/CMY, vanA1, vanA2, vanB, vanC1, or vanC2-C3-2.

Non-limiting examples of organisms that may be tested for and/or present in the polymicrobial infection include: one or a combination of: Acinetobacter baumannii, Actinotignum schaalii, Aerococcus urinae, Aerococcus urinae, Alloscardovia omnicolens, Candida albicans, Candida glabrata, Candida parapsilosis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhoeae, Pantoea agglomerans, Proteus mirabilis, Providencia stuartii, Pseudomonas aeruginosa, Serratia marcescens, Staphylococcus aureus, coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1&2, Adenovirus, or CMV.

In some embodiments, the therapeutic agent is one or a combination of a: penicillin, tetracycline, cephalosporin, quinolone, lincomycin, macrolide, sulfonamide, glycopeptide antibiotic, aminoglycoside, carbapenem, annamycin, lipopeptide, Fosfomycin, monobactam, nitrofuran, oxazolidinone, amphotericin B, Isavuconazole, itraconazole, micafungin, Posaconazole, voriconazole, cidofovir, vidarabine, foscarnet, acyclovir, valacyclovir, or a combination thereof.

In some embodiments, the polymicrobial infection comprises 2 or more organisms In some embodiments, the polymicrobial infection comprises 3 or more organisms. In some embodiments, the polymicrobial infection comprises 4 or more organisms. In some embodiments, the polymicrobial infection comprises 5 or more organisms. In some embodiments, the polymicrobial infection comprises 6 or more organisms. In some embodiments, the polymicrobial infection comprises 7 or more organisms.

In certain embodiments, the presence of two or more organisms in the polymicrobial infection changes sensitivity of the polymicrobial infection to antibiotics or therapeutic agents known to be effective against at least one of the organisms present in the polymicrobial infection individually.

The present invention also provides a workflow method for preparing a therapeutic solution for a patient having or suspected of having a polymicrobial infection. The method may comprise one or more of the steps described herein, for example subjecting a portion of the sample to a genetic identification testing, subjecting a portion of the sample to genetic resistance marker testing, and subjecting a portion of the sample to pooled antibiotic susceptibility testing.

In certain embodiments, the sample comprises urine, blood, plasma, cerebrospinal fluid, saliva, sputum, pulmonary lavage, vaginal secretions, wound lavage, biopsy tissue, wound swab, rectal swab, nasal swab, tissue, fecal matter, sperm sample, semen sample, or prostate fluid.

With reference to any of the embodiments herein, the steps described herein may be performed in any order, or simultaneously.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic view of mutualism between particular bacteria.

FIG. 2 shows the frequency of which certain resistance genes are found together. The strength of the correlation is represented by the width of the edge connecting the genes. Only correlations greater than 0.1 are shown.

FIG. 3 shows a comparison between the frequency polymicrobial infections are determined to be phenotypically sensitive yet genotypically resistant to an antibiotic (or vice versa), e.g., how often the phenotypic and genotypic sensitivity results disagree (n=764 symptomatic patients with polymicrobial infection and phenotype data).

FIG. 4 shows an example of a report comprising one or more therapeutic solutions for a particular polymicrobial infection.

FIG. 5 shows a comparison of symptom resolution in patients treated based on methods of the present invention (“Guidance UTI test”), patients treated empirically, and untreated patients.

FIG. 6 shows the numbers of patients testing positive to various bacteria by culture or PCR (all patients, n=2511).

FIG. 7 shows bacteria detected in polymicrobial infections in the study.

FIG. 8 shows the distribution of bacteria detected in the 68.6% (1710/2493) of patients who were positive for bacteria showing the distribution of organisms in monomicrobial, polymicrobial, and consortia mixtures.

FIG. 9 shows a network diagram of the relationships among the bacteria found most frequently in consortia

FIG. 10 shows detection rates for Gram-positive and Gram-negative bacteria in monomicrobial and polymicrobial infections and in consortia.

FIG. 11 shows the odds ratios of antibiotic resistance in polymicrobial versus monomicrobial specimens, along with the odds ratio of resistance for each increase in the number of bacterial species in polymicrobial specimens.

FIG. 12 shows the effects of organism interactions on antibiotic resistance. An upward arrow indicates an increase in resistance. A downward arrow indicates a decrease in resistance (an increase in sensitivity).

FIG. 13 shows the effects of E. coli and K. pneumoniae interactions on resistance to ampicillin/sulbactam, cefaclor, and tetracycline (p=0.05).

FIG. 14 depicts an exemplary Antibiotic Source Plate with well contents and antibiotic concentration (μg/mL). Nitro=nitrofurantoin, Cipro=ciprofloxacin, Mero=meropenem, Ceftiaxone=ceftriaxone, TMP/SMX=trimethoprim+sulfamethoxazole, Pip/Tazo=piperacillin+tazobactam, Levo=levofloxacin, Cefoxitin=cefoxitin, Tetra=tetracycline, Amp/Sulb=ampicillin+sulbactam, Amp=ampicillin, and Vanco=vancomycin.

FIG. 15 depicts an exemplary Antibiotic Source Plate with well contents and antibiotic concentration (μg/mL). Cefazolin=cefazolin, Cefepime=cefepime, Ceftazidime=ceftazidime, Gentamicin=gentamicin, Amox/Clav=amoxicillin+clavulanate, Cefaclor=cefaclor.

FIG. 16 shows the concordance between the presence of antibiotic resistance genes (ABR) detected by multiplex polymerase chain reaction (M-PCR) and antibiotic susceptibility detected by pooled antibiotic susceptibility testing (P-AST) of urine samples from symptomatic patients with urinary tract infection (UTI). The dashed line represents the weighted average concordance across all samples (60%). Abbreviation: Combo, combination antibiotics, including Ampicillin/Sulbactam, Amoxicillin/Clavulanate, and Piperacillin/Tazobactam.

FIG. 17 shows the number of patients (with either polymicrobial infections or monomicrobial infections) having sensitive or resistant ABR genes present for meropenem, piperacillin/tazobactam, or vancomycin.

FIG. 18 shows antibiotic resistance by pooled antibiotic susceptibility testing (P-AST) and antibiotic resistance (ABR) gene presence for all 14 antibiotics analyzed. Also shown are detection frequencies and associated average numbers of clinical findings per patient of different consortia detected in the study. Clinical findings are defined as one or more of the following symptoms or abnormal laboratory result, including urinary incontinence, dysuria, gross hematuria, pain/pelvic discomfort, urine cloudiness or strong smell, lower urinary tract symptoms (LUTS), and abnormal urinalysis or dipstick result.

FIG. 19 shows the overall concordance between the presence of antibiotic resistance (ABR) genes detected by multiplex polymerase chain reaction and antibiotic susceptibility detected using pooled antibiotic susceptibility testing (P-AST) of urine samples from symptomatic patients with urinary tract infection (UTI).

### TERMS

As used herein, the term “Highest Single Agent Interaction Principle” refers to a statistical model wherein the resistance of the polymicrobial infection is predicted to be the resistance of the bacteria with the highest resistance. For example, if species A is resistant with a probability 20%, and species B is resistant with a probability 50%, then the probability of resistance of the pool is 50%.

As used herein, the term “Union Principle” refers to a statistical model wherein the polymicrobial infection of species A and B is made up of one colony (or one genetic variant) of species A and one colony (or one genetic variant) of species B, and the polymicrobial infection is resistant if either the colony of species A is resistant, or if the colony of species B is resistant. For example, if an antibiotic is applied to the polymicrobial infection, it may kill off species A, but if species B survives, the polymicrobial infection is called resistant. For example, if species A is resistant with a probability 20%, and species B is resistant with a probability 50%, then the probability of resistance of the pool is: P (pool resistance)=P (A)+P(B)−P (A and B)

As used herein, the term “Logistic Additive Model” refers to a statistical model wherein the effects of species A and species B on the resistance of the polymicrobial infection is estimated in a logistic model. The effect of species A is the odds ratio of resistance when species A is present relative to when it is not present; similarly, the effect of species B is the odds ratio of resistance when species B is present relative to when it is not. The additive model predicts the effect of both species as the sum of the log odds-ratio; or the product of the two individual odds-ratios. For example, if the background resistance rate levofis 50%, the expected polymicrobial infection (species A and B) resistance with no interactions is 20%; if the background resistance rate is 20%, the expected polymicrobial infection resistance is 50%.

The present methods may be conducted using a plurality of antibiotics selected from the large number available to treat patients. Classes of antibiotics (also referred to as antimicrobial agents or anti-bacterial agents) include, but are not limited to, penicillins, tetracyclines, cephalosporins, quinolones, lincomycins, macrolides, sulfonamides, glycopeptide antibiotics, aminoglycosides, carbapenems, ansamycins, annamycins, lipopeptides, monobactams, nitrofurans, oxazolidinones, and polypeptides.

Penicillin antibiotics may include, but are not limited to, penicillin, methicillin, amoxicillin, ampicillin, flucloxacillin, penicillin G, penicillin V, carbenicillin, piperacillin, ticarcillin, oxacillin, dicloxacillin, azlocillin, cloxacillin, mezlocillin, temocillin, and nafcillin. Additionally, penicillin antibiotics are often used in combination with beta-lactamase inhibitors to provide broader spectrum activity; these combination antibiotics include amoxicillin/clavulanate, ampicillin/sulbactam, piperacillin/tazobactam, and clavulanate/ticarcillin. Tetracycline antibiotics include, but are not limited to, tetracycline, doxycycline, demeclocycline, minocycline, and oxytetracycline. Cephalosporin antibiotics may include, but are not limited to, cefadroxil, cephradine, cefazolin, cephalexin, cefepime, ceftaroline, loracarbef, cefotetan, cefuroxime, cefprozil, cefoxitin, cefaclor, ceftibuten, cetriaxone, cefotaxime, cefpodoxime, cefdinir, cefixime, cefditoren, ceftizoxime, cefoperazone, cefalotin, cefamanadole, ceftaroline fosamil, cetobiprole, and ceftazidime. Cephalosporin antibiotics are often used in combination with beta-lactamase inhibitors to provide broader spectrum activity; these combination antibiotics include, but are not limited to, avibactam/ceftazidime and ceftolozane/tazobactam. Quinolone antibiotics include, but are not limited to, lomefloxacin, ofloxacin, norfloxacin, gatifloxacin, ciprofloxacin, moxifloxacin, levofloxacin, gemifloxacin, cinoxacin, nalidixic acid, trovaloxacin, enoxacin, grepafloxacin, temafloxacin, and sparfloxacin. Lincomycin antibiotics may include, but are not limited to, clindamycin and lincomycin. Macrolide antibiotics may include, but are not limited to, azithromycin, clarithromycin, erythromycin, telithromycin, dirithromycin, roxithromycin, troleandomycin, spiramycin, and fidazomycin. Sulfonamide antibiotics may include, but are not limited to, sulfamethoxazole, sulfasalazine, mafenide, sulfacetamide, sulfadiazine, silver sulfadiazine, sulfadimethoxine, sulfanilamide, sulfisoxazole, sulfonamidochrysoidine, and sulfisoxazole. Sulfonamide antibiotics are often used in combination with trimethoprim to improve bactericidal activity. Glycopeptide antibiotics may include, but are not limited to, dalbavancin, oritavancin, telavancin, teicoplanin, and vancomycin. Aminoglycoside antibiotics may include, but are not limited to, paromomycin, tobramycin, gentamicin, amikacin, kanamycin, neomycin, netilmicin, streptomycin, and spectinomycin. Carbapenem antibiotics include, but are not limited to, imipenem, meropenem, doripenem, ertapenem, and imipenem/cilastatin. Ansamycin antibiotics may include, but are not limited to, geldanamycin, herbimycin, and rifaximin. Lipopeptide antibiotics may include, but are not limited to, daptomycin. Monobactam antibiotics may include, but are not limited to, aztreonam. Nitrofuran antibiotics may include, but are not limited to furazolidone and nitrofurantoin. Oxazolidinone antibiotics may include, but are not limited to, linezolid, posizolid, radezolid, and torezolid. Polypeptide antibiotics may include, but are not limited to, bacitracin, colistin, and polymyxin B.

Other antibiotics which are not part of any of the above-mentioned groups include, but are not limited to, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

Additionally, the scope of the presently disclosed methods encompasses the inclusion of antibiotics not yet known, or not yet approved by regulatory authorities. The presently claimed assay can be performed with any anti-bacterial agent and is not limited to the antibiotics disclosed herein.

### DETAILED DESCRIPTION OF THE INVENTION

### Therapeutic Solutions for Treatment of Polymicrobial Infections

Disclosed herein are methods and systems for identifying polymicrobial infections and identifying or providing therapeutic solutions for polymicrobial infections or suspected polymicrobial infections in a patient. The present invention also features methods and systems for treating polymicrobial infections or suspected polymicrobial infections in a patient.

The patient may be exhibiting symptoms of an infection. Non-limiting examples of symptoms include: fever, discharge, itching, dysuria, increased urinary frequency or urgency, etc. However, in some embodiments, the methods are applied to patients not experiencing symptoms of an infection. A patient, as used herein, refers to a mammal (e.g., human or other appropriate mammal).

The methods herein feature: (1) detection and identification of organisms (e.g., bacteria or other infectious agents such as viruses, fungi, protozoa, etc., a combination thereof) of the polymicrobial infection, (2) pooled antibiotic susceptibility tests for determining the susceptibility or resistance of the polymicrobial infection in the sample to an antibiotic or other therapeutic agent, and (3) identification of resistance genes in the infectious agents in the polymicrobial infection, e.g., genetic markers that may indicate resistance to a particular antibiotic or other therapeutic agent or treatment. Together, the data from (1), (2), and (3) can be applied (e.g., using databases of antibiotic/therapeutic susceptibility or resistance for particular known polymicrobial infections) in order to provide one or more therapeutic solutions for the polymicrobial infection.

The present invention is not limited to any particular type of infection. The present invention is not limited to bacterial infectious agents and may include viral infectious agents, fungal infectious agents, and/or protozoa as well.

Samples may be in the form of a fluid, biological matter, or tissue (e.g., biopsy). Samples may include but are not limited to blood samples, urine samples, plasma samples, saliva samples, pulmonary lavage samples, vaginal secretions, wound lavage, biopsy samples, wound samples, sperm samples, semen samples, prostate fluid samples, cerebrospinal fluid samples, synovial fluid samples, peritoneal fluid samples, pericardial fluid samples, pleural fluid samples, sputum samples, stool samples, mucosal samples, abscess samples, nasal samples, rectal samples, etc.

The present invention comprises subjecting a sample of a patient suspected of having an infection to an organism detection method for detecting and identifying organisms present in the sample. Quantitative polymerase chain reaction (PCR), which is well known to one of ordinary skill in the art, may be used to quickly detect organisms in the sample.

The organism detection method may allow for detection of a plurality of organisms, e.g., more than 5 organisms, more than 10 organisms, more than 20 organisms, more than 30 organisms, more than 40 organisms, more than 50 organisms, etc.

Samples identified as positive for infection-related organisms (e.g., UTI related organisms) are then tested to determine an antibiotic susceptibility profile for the infection. For example, the sample is subjected to phenotypic antibiotic susceptibility testing, e.g., pooled antibiotic susceptibility tests (P-AST), for determining the susceptibility or resistance of the infection to one more antibiotics or therapeutic agents, and genotypic antibiotic resistance testing (G-ABR) for determining the presence of antibiotic resistance genes, which may indicate resistance to a particular antibiotic or other therapeutic agent or treatment.

### Pooled Antibiotic Susceptibility Testing (P-AST)

The methods of the present invention further comprise pooled sensitivity tests wherein samples are incubated in plurality of antibiotics or other therapeutic agents. The samples are not first subjected to bacterial or organism isolation.

As an example, a sample may be fractioned and all or a portion of the fractions may be introduced to media containing an antibiotic or other therapeutic agent. P-AST tests may be performed in multi-well plates; however, the present invention is not limited to a multi-well plate format.

After incubation, the concentrations of the infectious agents (e.g., bacteria) are quantitated, thereby determining the susceptibility or resistance of the polymicrobial infection in the sample to the antibiotic or other therapeutic agent. For example, the media may be read in a spectrophotometer to determine the OD to measure cell density. The present invention is not limited to the use of a spectrophotometer to determine the OD to measure bacterial concentrations. Any other appropriate methods may be considered, e.g., fluorescence, chemiluminescence, etc. For example, in some embodiments, a color matrix system is utilized, wherein color change of an indicator is observed and/or calculated. In some embodiments, the present invention uses flow cytometry (which includes the use of antibodies to the organisms). In some embodiments, an electrical system is utilized. The present invention is not limited to the aforementioned methods.

In certain embodiments, P-AST features testing for 1 antibiotic or therapeutic agent. In certain embodiments, P-AST features testing for 2 or at least 2 antibiotics and/or therapeutic agents. In certain embodiments, P-AST features testing for 3 or at least 3 antibiotics and/or therapeutic agents. In certain embodiments, P-AST features testing for 4 or at least 4 antibiotics and/or therapeutic agents. In certain embodiments, P-AST features testing for 5 or at least 5 antibiotics and/or therapeutic agents. In certain embodiments, P-AST features testing for 6 or at least 6 antibiotics and/or therapeutic agents. In certain embodiments, P-AST features testing for 7 antibiotics and/or therapeutic agents. In certain embodiments, P-AST features testing for 8 or at least 8 antibiotics and/or therapeutic agents. In certain embodiments, P-AST features testing for 9 or at least 9 antibiotics and/or therapeutic agents. In certain embodiments, P-AST features testing for 10 or at least 10 antibiotics and/or therapeutic agents. In certain embodiments, P-AST features testing for 11 or at least 11 antibiotics and/or therapeutic agents. In certain embodiments, P-AST features testing for 12 or at least 12 antibiotics and/or therapeutic agents. In certain embodiments, P-AST features testing for 13 or at least 13 antibiotics and/or therapeutic agents. In certain embodiments, P-AST features testing for 14 or at least 14 antibiotics and/or therapeutic agents. In certain embodiments, P-AST features testing for 15 or at least 15 antibiotics and/or therapeutic agents. In certain embodiments, P-AST features testing for 16 or at least 16 antibiotics and/or therapeutic agents. In certain embodiments, P-AST features testing for 17 or at least 17 antibiotics and/or therapeutic agents. In certain embodiments, P-AST features testing for 18 or at least 18 antibiotics and/or therapeutic agents. In certain embodiments, P-AST features testing for 19 or at least 19 antibiotics and/or therapeutic agents. In certain embodiments, P-AST features testing for 20 or at least 20 antibiotics and/or therapeutic agents. In certain embodiments, P-AST features testing for 25 or at least 25 antibiotics and/or therapeutic agents. In certain embodiments, P-AST features testing for 30 or at least 30 antibiotics and/or therapeutic agents. In certain embodiments, P-AST features testing for 35 or at least 35 antibiotics and/or therapeutic agents. In certain embodiments, P-AST features testing for 40 or more than 40 antibiotics and/or therapeutic agents.

Non-limiting examples of antibiotics and other therapeutic agents are disclosed herein and are well known to one of ordinary skill in the art.

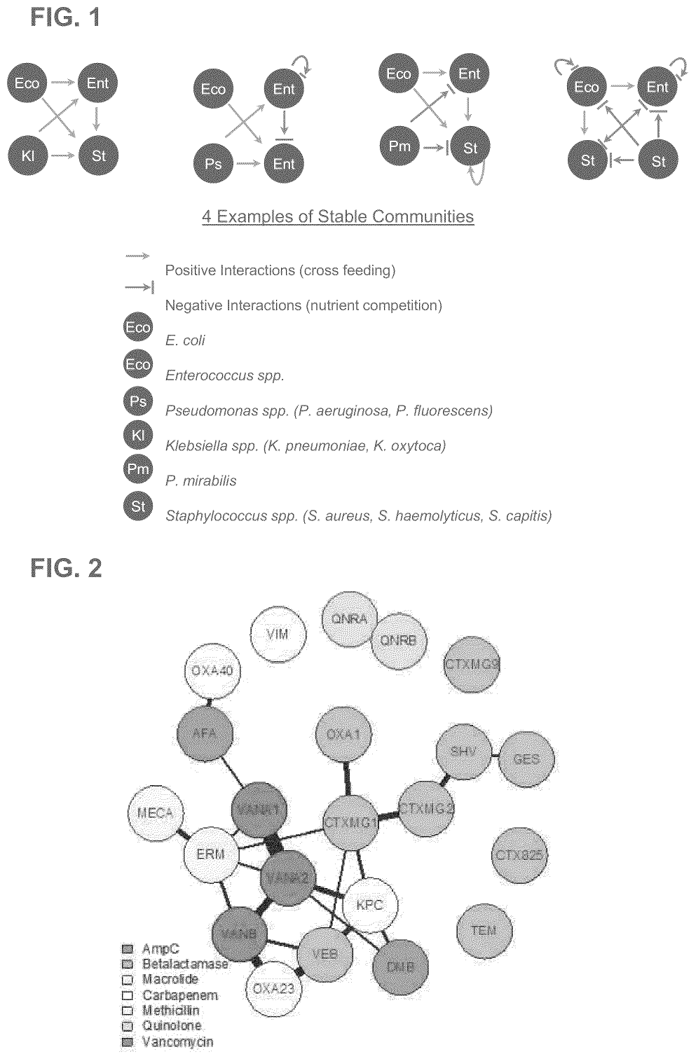

FIG. 1 describes the study of interactions of particular bacteria in polymicrobial infections subjected to pooled antibiotic sensitivity testing (P-AST). For example, one organism (e.g., metabolite) can feed another; in some examples, some organisms may protect others from particular antibiotics or make them more susceptible to particular antibiotics.

### Genetic Antibiotic Resistance Testing (G-ABR)

Table 1 below lists non-limiting examples of genes tested in genetic antibiotic resistance testing (G-ABR). The presence of one or more genes below may confer resistance to AmpC, Carbapenem, Extended-Spectrum Beta-lactamase, Macrolide, Methicillin, Quinolone, Fluoroquinolone, or Vancomycin.

TABLE 1

| Gene | Category |
|---|---|
| ampC, FOX, ACC | Ampicillin resistance |
| DHA, MOX/CMY, | |
| BIL/LAT/CMY | |
| blaOXA-48 | Carbapenem resistance |
| IMP-1 group | |
| IMP-7 | |
| KPC | |
| NDM | |
| OXA-23 | |
| OXA-40 | |
| OXA-48 | |
| OXA-58 | |
| OXA-72 | |
| VIM | |
| NDM-1 | Extended-Spectrum-Betalactamase |
| CTX-M group 1 | |
| CTX-M group 2 | |
| CTX-M group 8/25 | |
| CTX-M group 9 | |
| GES | |
| OXA-1 | |
| PER-1 | |
| PER-2 | |
| SHV | |
| TEM | |
| VEB | |
| ErmA + ErmB | Macrolide resistance |
| mecA | Methicillin resistance |
| QnrA | Quinolone and fluoroquinolone resistance |
| QnrB | |
| vanA1 | Vancomycin resistance |
| vanA2 | |
| vanB | |

In certain embodiments, G-ABR features testing for 1 genetic marker (e.g., antibacterial resistance gene, antiviral resistance gene, antifungal resistance gene) indicating resistance to a particular therapeutic agent. In certain embodiments, G-ABR features testing for 2 or at least 2 genetic markers (e.g., antibacterial resistance genes, antiviral resistance genes, antifungal resistance genes) indicating resistance to particular therapeutic agents. In certain embodiments, G-ABR features testing for 3 or at least 3 genetic markers (e.g., antibacterial resistance genes, antiviral resistance genes, antifungal resistance genes) indicating resistance to particular therapeutic agents. In certain embodiments, G-ABR features testing for 4 or at least 4 genetic markers (e.g., antibacterial resistance genes, antiviral resistance genes, antifungal resistance genes) indicating resistance to particular therapeutic agents. In certain embodiments, G-ABR features testing for 5 or at least 5 genetic markers indicating resistance to particular therapeutic agents. In certain embodiments, G-ABR features testing for 6 or at least 6 genetic markers indicating resistance to particular therapeutic agents. In certain embodiments, G-ABR features testing for 7 or at least 7 genetic markers indicating resistance to particular therapeutic agents. In certain embodiments, G-ABR features testing for 8 or at least 8 genetic markers indicating resistance to particular therapeutic agents. In certain embodiments, G-ABR features testing for 9 or at least 9 genetic markers indicating resistance to particular therapeutic agents. In certain embodiments, G-ABR features testing for 10 or at least 10 genetic markers indicating resistance to particular therapeutic agents. In certain embodiments, G-ABR features testing for 11 or at least 11 genetic markers indicating resistance to particular therapeutic agents. In certain embodiments, G-ABR features testing for 12 or at least 12 genetic markers indicating resistance to particular therapeutic agents. In certain embodiments, G-ABR features testing for 13 or at least 13 genetic markers indicating resistance to particular therapeutic agents. In certain embodiments, G-ABR features testing for 14 or at least 14 genetic markers indicating resistance to particular therapeutic agents. In certain embodiments, G-ABR features testing for 15 or at least genetic markers indicating resistance to particular therapeutic agents. In certain embodiments, G-ABR features testing for 16 or at least 16 genetic markers indicating resistance to particular therapeutic agents. In certain embodiments, G-ABR features testing for 17 or at least 17 genetic markers indicating resistance to particular therapeutic agents. In certain embodiments, G-ABR features testing for 18 or at least 18 genetic markers indicating resistance to particular therapeutic agents. In certain embodiments, G-ABR features testing for 19 or at least 19 genetic markers indicating resistance to particular therapeutic agents. In certain embodiments, G-ABR features testing for 20 or at least 20 genetic markers indicating resistance to particular therapeutic agents. In certain embodiments, G-ABR features testing for 25 or at least 25 genetic markers indicating resistance to particular therapeutic agents. In certain embodiments, G-ABR features testing for 30 or at least 30 genetic markers indicating resistance to particular therapeutic agents. In certain embodiments, G-ABR features testing for 35 or at least 35 genetic markers indicating resistance to particular therapeutic agents. In certain embodiments, G-ABR features testing for 40 or at least 40 genetic markers indicating resistance to particular therapeutic agents. In certain embodiments, G-ABR features testing for more than 40 genetic markers indicating resistance to particular therapeutic agents.

Non-limiting examples of genetic markers (e.g., antibacterial resistance genes, antiviral resistance genes, antifungal resistance genes) are disclosed herein and are well known to one of ordinary skill in the art.

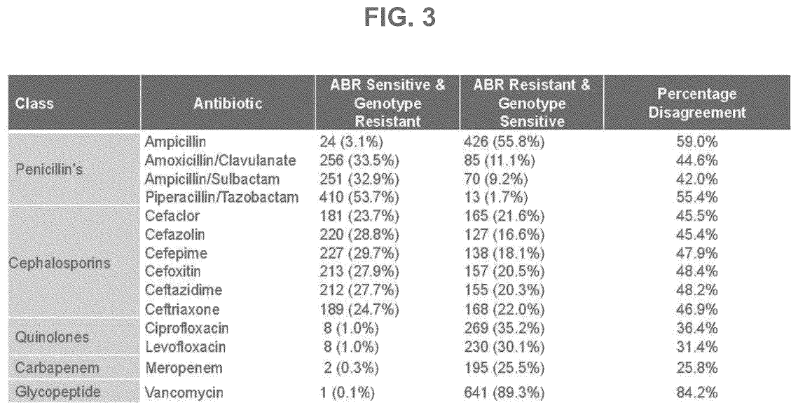

FIG. 2 shows the results of a study wherein samples were tested for the presence of resistance genes and the frequency that particular resistance genes were found together. FIG. 3 shows the results of a study wherein a collection of samples were evaluated for ABR status (sensitivity or resistance) and tested for resistance markers (resistant genotype or a sensitive genotype) with respect to one or more antibiotics. Notably, there were instances where the two results were in disagreement, e.g., instances wherein the organisms are phenotypically sensitive to an antibiotic yet have a resistant genotype (see column 3) and instances wherein the organisms are phenotypically resistant yet have a sensitive genotype (see column 4). This may be used to help determine the likelihood of phenotypic sensitivity or resistance of even when the organism(s) can't be grown in culture.

Together, the P-AST data and G-ABR data can be analyzed (e.g., applied or compared to standardized values or thresholds) for determining antibiotic/therapeutic susceptibility or resistance in order to provide one or more therapeutic solutions for the polymicrobial infection. Thresholds will be specific to each test, e.g., for the genetic identification testing, pooled sensitivity testing, resistance gene testing. For example, a threshold for genetic identification testing may include a minimum number of organisms to test positive for the organism. Note that the present invention also includes the testing of resistance genes that will be identified in the future. The present invention also includes the culturing of bacteria that presently cannot be cultured but may be cultured in the future as growth conditions are expanded.

In certain embodiments, the therapeutic agent is one or a combination of a: penicillin, tetracycline, cephalosporin, quinolone, lincomycin, macrolide, sulfonamide, glycopeptide antibiotic, aminoglycoside, carbapenem, ansamycin, annamycin, lipopeptide, Fosfomycin, monobactam, nitrofuran, oxazolidinone, and/or a polypeptide. In certain embodiments, the therapeutic agent is one or a combination of cidofovir, vidarabine, foscarnet, acyclovir, and/or valacyclovir. In certain embodiments, the therapeutic agent is one or a combination of amphotericin B, isavuconazole, itraconazole, micafungin, Posaconazole, and/or voriconazole.

Formulations of the therapeutic agents include but are not limited to oral (PO) intravenous, (IV), and/or injection.

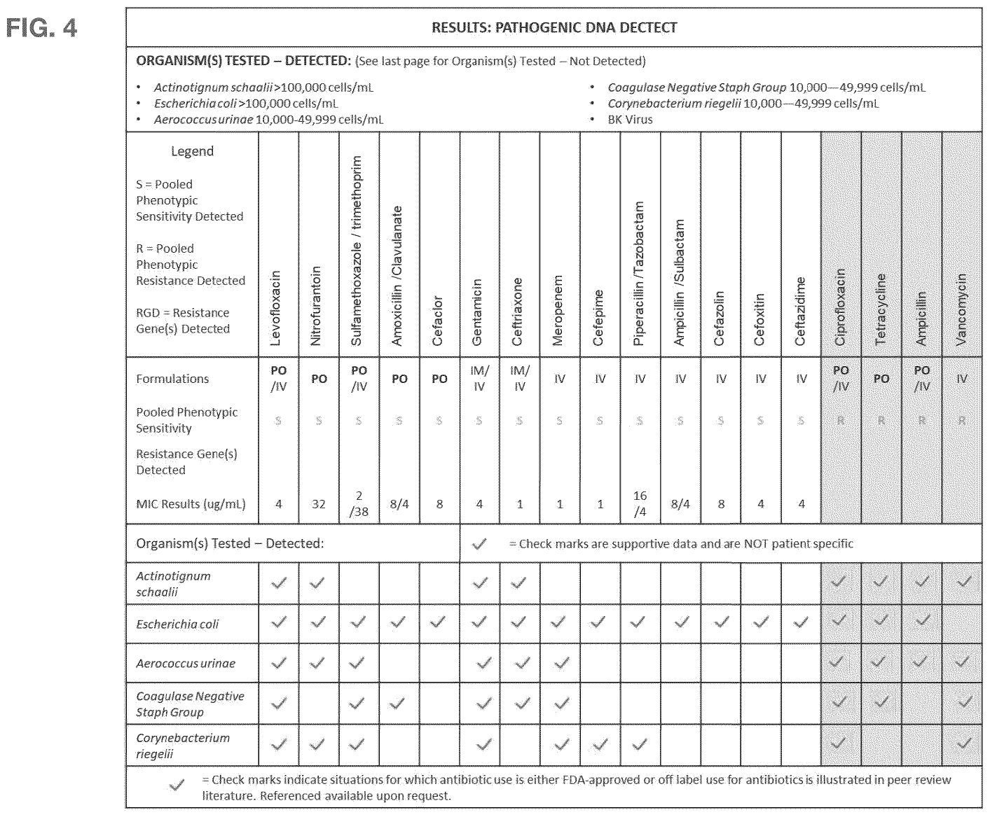

FIG. 4 shows an example of a report describing the results of the methods of the present invention for a sample of a polymicrobial infection, including one or more therapeutic solutions.

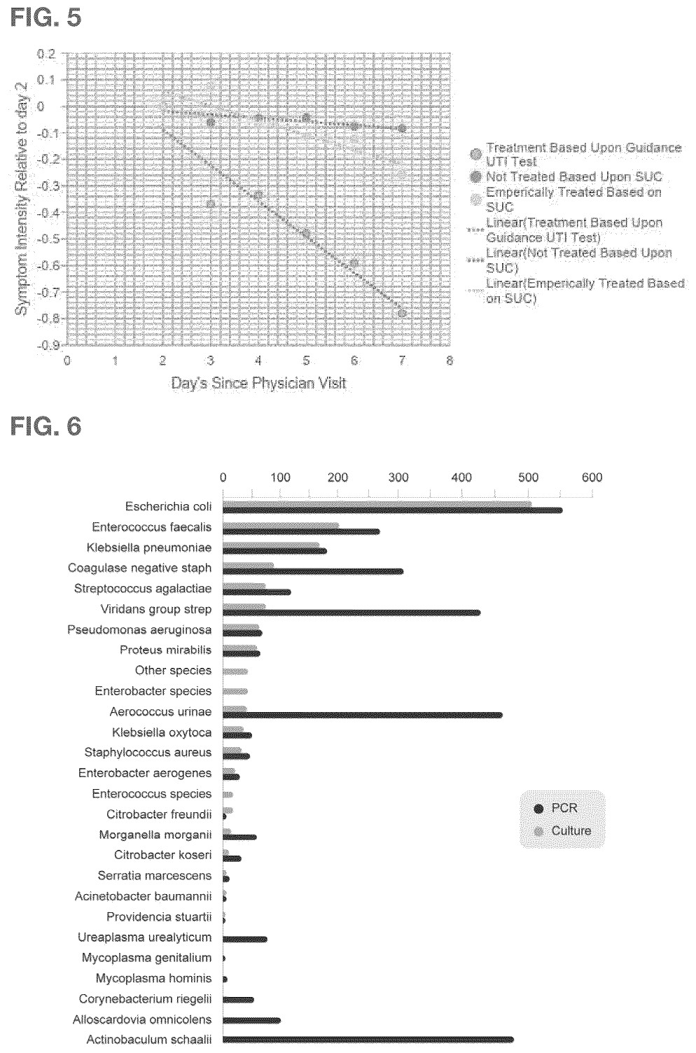

The methods herein help provide fast results and reduce the need for empiric therapy. Without wishing to limit the present invention to any theory or mechanism, the methods of the present invention are believed to provide better accuracy compared to current standard of care practices such as standard urine culture. Referring to FIG. 5, patients treated using the methods of the present invention (“Guidance UTI test”) showed significantly better symptom resolution as compared to patients untreated or patients treated empirically.

### EXAMPLES

The following are non-limiting examples of the present invention. It is to be understood that said examples are not intended to limit the present invention in any way. Equivalents or substitutes are within the scope of the present invention.

### Example 1. Multisite Prospective Comparison of Multiplex Polymerase Chain Reaction Testing with Urine Culture for Diagnosis of Urinary Tract Infections in Symptomatic Patients

Urine culture is traditionally used for detection and identification of pathogens for diagnosis and management of urinary tract infections (UTIs). However, a growing body of evidence suggests that polymerase chain reaction (PCR) testing may be superior for both detection and identification of pathogens. This prospective multicenter study compared PCR with traditional urine culture for detection and identification of bacteria in 2511 patients (mean age 73; range 24-100) presenting with symptoms of UTI. Both urine cultures and PCR were performed on samples from all patients. PCR detected bacteria in 62.7% (1575/2511) of cases, while urine culture detected bacteria in 43.7% (1098/2511) of cases. PCR and culture agreed in 74.6% of cases: both were positive in 40.5% (1018/2511) and both were negative in 34.1% (856/2511). PCR and culture disagreed in 25.4% of cases: PCR was positive while culture was negative in 22.2% (557/2511) and PCR was negative while culture was positive in 3.2% (80/2511). A total of 861 polymicrobial infections were reported, with PCR reporting 834 (96.9%) and culture reporting 168 (19.5%). Polymicrobial infections were reported in 268 patients (10.7%) in which culture results were negative. Culture completely missed five clinically significant bacterial pathogens. Missed bacterial detection can result in inadequate consideration of bacterial resistance and susceptibility, which can then lead to treatment failure. The greater sensitivity and specificity of PCR may afford opportunities to treat UTI more effectively.

Study participants were patients who presented with symptoms of UTI at urology clinics. The patients were evaluated by any of 75 physicians from 37 urology offices in seven states. A total of 2511 consecutive patients who met inclusion criteria were enrolled between Jul. 26, 2018 and Feb. 27, 2019. No predetermined quotas or ratios for gender participation of male and female subjects were imposed.

Inclusion criteria: Patients presenting with symptoms of acute cystitis, complicated UTI, persistent UTI, or recurrent UTIs, or prostatitis, pyelonephritis, and/or interstitial cystitis; Symptoms of interstitial cystitis at any age; Symptoms of other conditions at 60 years of age; Specimen volumes sufficient volume to permit urine culture and Guidance 4.0; Patient informed consent; Documented times at which the specimen samples were collected and stabilized.

Exclusion criteria: Prior participation in this study; Taking antibiotics for any reason other than UTI at the time of enrollment; Chronic (≥10 days) indwelling catheters; Self-catheterization; Patients with urinary diversion; Absence of written informed consent and/or HIPAA authorization form.

Urine samples were obtained from patients either by self-administered clean catch or by catheterization. The samples were collected and transported to Pathnostics (Irvine, California) for testing by culture. For culture, urine was vortexed, and a sterile plastic loop (1 uL) was used to inoculate blood agar plates. A sterile plastic loop (1 uL) was used also to inoculate colistin and nalidixic acid agar/MacConkey agar (CNA/MAC) plates, one loop-full of urine on the CNA side of the plate and another full loop-full on the MAC side of the plate. All plates were incubated at 35° C. in 5% CO₂ for ≥18 hours, then examined for evidence of growth. Plates with <10⁴ CFU/ml were reported as normal urogenital flora. For plates with growth (≥10⁴ CFU/ml), the quantity and morphology of each organism was recorded. The maximum readable colony count using the 1 uL loop is >10⁵ CFU/ml. Colony counts were performed on the blood agar plates. Species identification and colony counts were performed on CNA/MAC plates. For plates with ≤2 pathogens, species identification and colony counts were reported for each pathogen with ≥10⁴ CFU/ml. If ≥3 pathogens were present, and one or two were predominant, species identification and colony counts were reported. If ≥3 pathogens were present without predominant species, a mixed morphotype was reported.

Pathogen identification was confirmed with the VITEK 2 Compact System (bioMerieux, Durham, NC) in accordance with standard operating procedures. Briefly, a sterile swab was used to transfer morphologically similar colonies from positive blood agar plates to prepared polystyrene test tubes containing 3.0 mL of sterile saline. The sample was adjusted for density (equivalent to McFarland No. 0.50 to 0.63). The sample tube and an appropriate identification card were placed into the cassette and inserted into the VITEK 2 instrument. The identity of the bacteria was used to determine Gram status, and a GN card was used for Gram-negative bacteria, and a GP card was used for Gram-positive bacteria. A YST card was used for yeast. Pathogen identification was read from the VITEK 2 instrument.

DNA was extracted from urine samples with the KingFisher/MagMAX Automated DNA Extraction instrument and the MagMAX DNA Multi-Sample Ultra Kit (ThermoFisher, Carlsbad, CA). Briefly, 400 uL of urine were transferred to wells in 96-well deep-well plates, sealed, and centrifuged to concentrate the samples; supernatant was removed. Enzyme Lysis Mix (220 uL/well) was added and incubated for 20 min at 65° C. Proteinase K Mix (PK Mix) was added (50 uL/well) and incubated for 30 min at 65° C. Lysis buffer (125 μL/well) and DNA Binding Bead Mix (40 μL/well) were added, and the samples shaken for a minimum of 5 min. The 96-well plate was loaded into the KingFisher/MagMAX Automated DNA Extraction instrument, which was operated in accordance with standard operating procedures.

DNA samples were analyzed with the Pathnostics Guidance™ UTI Test. The samples were mixed with universal PCR master mix and amplified with TaqMan technology on a Life Technologies 12K Flex Open Array System. DNA samples were spotted in duplicate on 112-format OpenArray chips. Plasmids for each organism being tested for were used as positive controls. Candida tropicalis was used as an inhibition control. A data analysis tool developed by Pathnostics was used to sort data, assess the quality of data, summarize control sample data, identify positive assays, calculate concentrations, and generate draft reports. Probes and primers were used for the following pathogens: Bacteria: Acinetobacter baumannii, Actinotignum schaalii, Aerococcus urinae, Alloscardovia omnicolens, Citrobacter freundii, Clostridium difficile, Citrobacter koseri, Corynebacterium riegelii, Enterobacter aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Pantoea agglomerans, Proteus mirabilis, Providencia stuartii, Pseudomonas aeruginosa, Serratia marcescens, Staphylococcus aureus, Streptococcus agalactiae, and Ureaplasma urealyticum. Bacterial groups: Coagulase negative staphylococci (Staphylococcus epidermidis, Staphylococcus haemolyticus, Staphylococcus lugdunensis, Staphylococcus saprophyticus); Viridans group streptococci (Streptococcus anginosus, Streptococcus oralis, Streptococcus pasteuranus).

Demographics and symptoms were compared for male and female patients with two-sample t-tests or Fisher's exact tests, as appropriate. The proportion of samples testing positive for specific bacteria were compared for PCR and culture results using McNemar's test. Of the 2511 participants, 1360 (54%) were females and 1151 (46%) were males, with ages 24 years to 100 years. Dysuria, urinary incontinence, cloudy or strong-smelling urine, low-grade fever, and pain in the abdomen, flank, low back or pelvis were significantly more frequent in women than in men. Pain was more severe in women than in men. Among lower urinary tract symptoms, urinary frequency and urgency were significantly more common in women, whereas nocturia and microhematuria were more common in men. Women had significantly more of these symptoms or signs than did men: acute change in mentation; increased falls or clumsiness; tiredness; feeling ill; and decline in activities of daily living. Increased numbers of leukocytes and concentrations of nitrites were present in the urine of women more often than in the urine of men, and UTIs were treated with antibiotics significantly more often in women than in men.

Table 2 (below) illustrates the percentage agreement (or disagreement) between traditional urine culture and PCR in patients with UTI symptoms. In 1874 of the 2511 (74.6%) cases, results of the two tests were in agreement; culture and PCR were both positive in 1018/2511 (40.5%) cases and both were negative in 856/2511 (34.1%) cases. PCR was positive whereas culture was negative in 557/2511 (22.2%) cases, and PCR was negative whereas culture was positive in 80/2511 (3.2%) of cases. Culture did not detect six bacterial species that were detected by PCR: Actinotignum schaalii, Alloscardovia omnicolens, Corynebacterium riegelii, Mycoplasma genitalium, Mycoplasma hominis, and Ureaplasma urealyticum). These six bacteria constituted 9.0% (67/741) of monomicrobial infections, but were detected in 60.7% (523/861) of polymicrobial infections.

TABLE 2
Agreement of PCR and traditional urine culture in patients
with symptoms of UTI

| | PCR Positive | PCR Negative | Total |
|---|---|---|---|
| Culture Positive | 40.5% | 3.2% | 43.7% |
| Culture Negative | 22.2% | 34.1% | 56.3% |
| Total | 62.7% | 37.3% | 100% |

PCR did not detect three bacteria that were detected by culture (FIG. 6). That PCR did not detect these bacteria is not a failure of the PCR because the PCR panel did not include probes for them.

The multiplex panel used in this prospective study tested for 24 bacteria plus two bacterial groups. PCR detected 24 different bacteria and culture detected 21 different bacteria.

The failure of culture to detect six bacteria produces a very different bacterial profile from that produced by PCR (see Table 3).

TABLE 3

| Rank | | | | |
|---|---|---|---|---|
| Order | By Culture Percentage | | By PCR Percentage | |
| 1 | Escherichia coli | 42.1% | Escherichia coli | 18.0% |
| 2 | Enterococcus faecalis | 15.3% | Actinotignum schaalii | 15.2% |
| 3 | Klebsiella pneumoniae | 11.1% | Aerococcus urinae | 14.5% |
| 4 | Coagulase-negative | 5.7% | Viridans group | 13.9% |
| | staphylococci | | streptococci | |
| 5 | Streptococcus | 4.3% | Coagulase-negative | 9.8% |
| | agalactiae | | staphylococci | |
| 6 | Viridans group | 4.0% | Enterococcus faecalis | 8.0% |
| | streptococci | | | |
| 7 | Pseudomonas | 3.0% | Klebsiella pneumoniae | 4.6% |
| | aeruginosa | | | |
| 8 | Proteus mirabilis | 3.0% | Alloscardovia omnicolens | 3.4% |
| 10 | Enterobacter species | 1.8% | Streptococcus agalactiae | 3.2% |

The only similarity between the rank order for culture and for PCR is that E. coli is the most frequently detected bacterium for both. However, E. coli constituted 42.1% of bacteria detected by culture, whereas E. coli constituted only 18% of bacteria detected by PCR. The number of patients in whom E. coli was detected was not much different between culture (532 patients) and PCR (570 patients), although the agreement was statistically different (McNemar's test p-value=0.0002). However, since PCR detected more different types of bacteria and a larger number of each type of bacteria, E. coli constituted a smaller percentage. For instance, K. pneumoniae was the third most frequently detected bacterium in culture (11.1%), but it was the seventh most frequently detected bacterium by PCR (4.6%). The number of patients in whom K. pneumoniae was detected was higher for PCR (145) than it was for culture (140) but the agreement for this species was not statistically different for PCR and culture (McNemar's test p-value=0.50).

In this study, culture was found to have limited capacity to detect and identify polymicrobial infections (see Table 4 below).

TABLE 4
Detection Rates for Polymicrobial Infections

| | | PCR | | | |
|---|---|---|---|---|---|
| | | | Mono- | | |
| | | Polymicrobial | microbial | Negative | Total |
| Culture | Poly- | 5.6% | 1.0% | 0.1% | 6.7% |
| | microbial | | | | |
| | Mono- | 16.9% | 17.0% | 3.1% | 37.0% |
| | microbial | | | | |
| | Negative | 10.7% | 11.5% | 34.1% | 56.3% |
| | Total | 33.2% | 29.5% | 37.3% | 100.0% |

Eight hundred sixty-one polymicrobial infections were detected by PCR and culture combined. This was 34.3% (861/2511) of the total patients and 52% (861/1655) of total positive results. PCR detected 834 of 861 (96.9%), and culture detected 168 of 861 (19.5%); 141 of 861 cases (16.4%) cases were polymicrobial by both PCR and culture. Polymicrobial infections constituted 53.0% of PCR positives (534/1575) but only 15.3% of culture positives (168/1098).

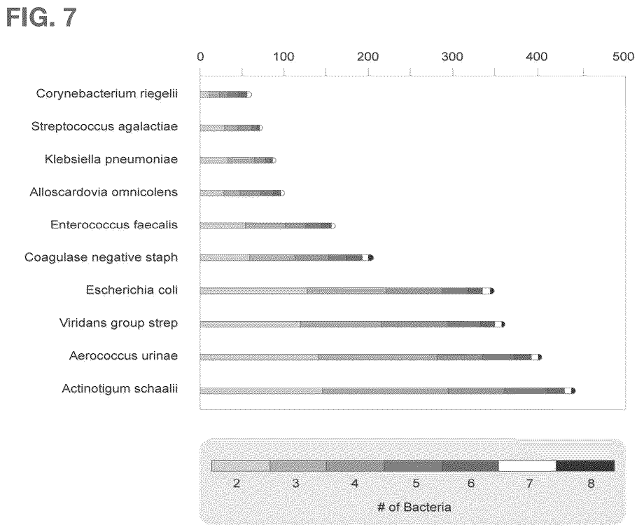

In this study, six bacteria that were detected by PCR were not detected by culture. Three of these bacteria (Actinotignum schaalii, Alloscardovia omnicolens, and Corynebacterium riegelii) were among the 10 most frequently detected bacteria, combining for 20.6% of all bacterial detections. Even more, Actinotignum schaalii was the bacterium involved in polymicrobial infections more often than any other and was involved in 53.0% (442/834) of all polymicrobial infections (FIG. 7).

This prospective study of over 2500 patients with symptoms of UTI found that multiplex PCR was positive in 62.7% of patients versus 43.7% with traditional urine culture. Twenty-two percent of PCR-positive patients had a negative urine culture and PCR detected five times more polymicrobial infections than did urine culture (834 vs. 168). Thus, the results of this study provide evidence that PCR affords greater detection sensitivity and identification specificity for diagnosis of bacterial UTI than does traditional culture.

The superiority of PCR over traditional culture was especially evident in the detection of polymicrobial infections: In 861 total cases, PCR detected 834, whereas culture detected 168. Culture is inherently limited in its ability to detect and resolve the identity of pathogens in polymicrobial infections. Although there are several reasons for the limitations of culture, a significant factor is the inability of some bacteria to be cultured. In this study, six bacteria that were detected by PCR were not detected by culture. Three of these bacteria (Actinotignum schaalii, Alloscardovia omnicolens, and Corynebacterium riegelli) were among the 10 most frequently detected bacteria, combining for 20.6% of all bacterial detections. Even more, Actinotignum schaalii was the bacterium involved in polymicrobial infections more often than any other and was involved in 53.0% (442/834) of all polymicrobial infections.

There is ample evidence to support the clinical relevance of all six of the bacteria detected by PCR but not urine culture. All six were detected at levels well above a threshold of 10⁴ CFU/mL. In fact, the lowest median detection level for any of these six bacteria was 2.1×10 4 CFU/mL for Corynebacterium riegelli, and five of the six bacteria were detected at levels in excess of 100,000 CFU/mL; C. riegelli is the exception. Five of the six have also been reported as pathogenic for UTI. Mycoplasma genitalium is the exception here. M. genitalium is a sexually transmitted infection that can cause urethritis in both men and women. However, up to 25% of men and 13% of women are infected with M. genitalium but remain asymptomatic, and detection may be cause to consider treatment. Of the others, Actinotignum schaalii and Alloscarovia omnicolens are acknowledged as uropathogens. Corynebacteria riegelli is commonly regarded as a commensal, but has also been shown to cause UTI. And both Mycoplasma hominis and Ureaplasma urealyticum have been documented as causing lower urinary tract symptoms, and may be antibiotic resistant.

Failure to detect and identify these bacteria raises the possibility of treatment failure due to unidentified antibiotic resistance. For instance, Ureaplasma urealyticum has no peptidoglycan layer in its cell wall and does not synthesize folate, which means that it is resistant to most available antibiotics. Several papers have reported that clinical antibiotic resistance (antibiotic response in the patient) was different from the resistance predicted based on the results of laboratory tests. This difference could occur for a number of reasons, but the use of isolated bacterial cultures for susceptibility testing means that failure to detect bacteria will lead to failures of susceptibility testing.

As an example of this problem, culture detects Klebsiella pneumoniae, but it does not detect Actinotignum schaalii; in this study, both bacteria are ranked second in occurrence in culture as well as PCR even though the two bacteria have very different resistance and susceptibility profiles; in some ways, their resistance/susceptibility profiles are mirror images of each other, and detection of K. pneumoniae but not A. schaalii could lead to selection of inappropriate antibiotics. For instance, culture detected Klebsiella pneumoniae, but not Actinotignum schaalii. Because K. pneumoniae and A. schaalii share no commonalities with respect to antibiotic resistance and susceptibility, selection of an antibiotic regimen on the basis of detection of K. pneumoniae alone would result in treating with an antibiotic that A. schaalii is resistant to.

Failure to appropriately treat UTI increases the risk of developing additional medical problems, some of which may be even more severe than recurrent UTI. Although many kinds of infections are associated with acute ischemic stroke, UTI presents the greatest risk, with an odds ratio of 5.32. The seven bacteria that were missed by culture but detected by PCR in this study have been associated with increased risk for sepsis and bacteremia, endocarditis, Fournier's gangrene, and abdominal abscess.

The proportion of the total number of patients with polymicrobial infections in this study was 34.3%. However, an alternative perspective is that among patients who tested positive for a bacterial UTI, there was a greater number of polymicrobial infections (52%, 861/1655) than monomicrobial infections (48%, 794/1655).

All bacteria do not contribute equally to pathogenesis. This is especially true for polymicrobial infections: some bacteria may be more pathogenic than others.

The results of this prospective study of over 2500 patients with symptoms of UTI demonstrated the greater detection sensitivity and identification specificity of PCR over traditional urine culture, especially in the detection of polymicrobial infections. Use of PCR in the evaluation of UTIs might improve the detection of pathogenic bacteria and consideration of antibiotic resistance and susceptibility. These advantages in turn might lead to more effective initial treatment and prevention of recurrent UTIs.

### Example 2. Bacterial Consortia Contribute to Pathogenesis of Polymicrobial Urinary Tract Infections

Urine is not sterile and has a unique microbiome, referred to as the urobiome. The view that urine is sterile arises from limitations of Standard Urine Culture (SUC), developed to detect common pathogens, such as uropathogenic Escherichia coli. However, SUC-independent methods, such as polymerase chain reaction (PCR), have demonstrated that culture fails to detect the majority of bacteria in urine, even in healthy individuals. Furthermore, expanded quantitative urine culture methods, which enhance the growth conditions for cultivating bacteria, show that the organisms uniquely detected by molecular methods are viable. The capability to detect and identify organisms in the urobiome in both health and disease has allowed a more detailed evaluation of dysbiosis as a potential mechanism for the cause of urinary tract infections (UTI).

Dysbiosis can take three forms, all of which involve a shift in the balance of bacteria in a microbiome. One type of dysbiosis is referred to as gain of function dysbiosis, in which there is an overgrowth of pathogens that can cause disease. A second type is known as loss of function, in which bacteria that would otherwise function to protect health are lost. Loss of function dysbiosis is often associated with use of antibiotics, resulting in the loss of both beneficial and pathogenic organisms. The third type of dysbiosis is a mixture of gain and loss of function.

This analysis investigated patterns of gain of function dysbiosis that may contribute to the pathogenesis of polymicrobial UTI. Formation of bacterial consortia and biofilms can cause gain of function dysbiosis. Consortia are non-random polymicrobial communities that interact synergistically, providing community members with growth and survival advantages over planktonic, free-floating, microbes. Like biofilms, consortia are self-organizing structures, and consortia can attach to surfaces to become biofilms. Multiple consortia may be found within individual biofilms, separated by interstitial spaces filled with fluid. However, biofilms can be monomicrobial, whereas consortia are inherently polymicrobial.

Many, if not most, infections are polymicrobial. However, not all polymicrobial infections form either consortia or biofilms. Those that do are likely to be more virulent and resistant to treatment. A better understanding of relationships between bacteria that constitute consortia in UTIs may contribute to improved diagnosis and interventional strategies. This study attempts to characterize bacterial consortia in UTI through retrospective analysis of prospective data from patients seen in multiple busy urology clinics with symptoms of a UTI. Secondly, this study attempts to identify possible symptom patterns associated with the identified bacterial consortia.

Consortia, as used herein, may be defined as non-random patterns of bacterial communities that are found together in symptomatic patients.

This study is a retrospective analysis of data gathered for a prospective study regarding detection rates for bacteria in UTI. Study participants consisted of 2493 patients who presented at any of 37 geographically different urology clinics in the United States between July 2018 and February 2019. All patients provided written informed consent per forms approved by the Western IRB (20181661). All subjects met the following inclusion and exclusion criteria. Inclusion criteria included: ≥60 years of age; symptoms of acute cystitis, complicated UTI, persistent UTI, recurrent UTI, prostatitis, pyelonephritis, or interstitial cystitis; specimen volumes sufficient to permit urine culture and multiplex polymerase chain reaction (M-PCR); documented times at which the specimens were collected and stabilized with boric acid in grey-top tubes. Exclusion criteria included antibiotics taken for any reason other than UTI at the time of enrollment, chronic (≥10 days) indwelling catheters, self-catheterization, and urinary diversion. Physicians recorded ICD-10 codes for each clinical encounter, as well as the patients' presenting UTI symptoms and urinalysis dipstick results. Catheterized and clean catch urine specimens were collected, depending on the patients' ability.

DNA was extracted from urine samples with the KingFisher/MagMAX Automated DNA Extraction instrument and the MagMAX DNA Multi-Sample Ultra Kit (ThermoFisher, Carlsbad, CA). Briefly, 400 uL of urine were transferred to wells in 96-well deep-well plates, sealed, and centrifuged to concentrate the samples; supernatant was removed. Enzyme Lysis Mix (220 uL/well) was added and incubated for 20 min at 65° C. Proteinase K Mix was added (50 uL/well) and incubated for 30 min at 65° C. Lysis buffer (125 μL/well) and DNA Binding Bead Mix (40 μL/well) were added, and the samples shaken for a minimum of 5 min. The 96-well plate was loaded into the KingFisher/MagMAX Automated DNA Extraction instrument, which was operated in accordance with standard operating procedures.

DNA samples were analyzed with the Pathnostics Guidance™ UTI Test. The samples were mixed with universal PCR master mix and amplified with TaqMan technology on a Life Technologies 12K Flex Open Array System. DNA samples were spotted in duplicate on 112-format OpenArray chips. Plasmids for each organism being tested for were used as positive controls. Candida tropicalis was used as an inhibition control. A data analysis tool developed by Pathnostics was used to organize data, assess the quality of data, summarize control sample data, identify samples with positive assay results, calculate concentrations, and generate draft reports. Probes and primers were used for the following pathogens:

Bacteria: Acinetobacter baumannii, Actinotignum schaalii, Aerococcus urinae, Alloscardovia omnicolens, Citrobacter freundii, Clostridium difficile, Citrobacter koseri, Corynebacterium riegelii, Enterobacter aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Pantoea agglomerans, Proteus mirabilis, Providencia stuartii, Pseudomonas aeruginosa, Serratia marcescens, Staphylococcus aureus, Streptococcus agalactiae, and Ureaplasma urealyticum.

Bacterial groups: Coagulase negative staphylococci (Staphylococcus epidermidis, Staphylococcus haemolyticus, Staphylococcus lugdunesis, Staphylococcus saprophyticus); Viridans group streptococci (Streptococcus anginosus, Streptococcus oralis, Streptococcus pasteuranus).

In order to differentiate between consortia and random associations of bacteria, a cutoff of 10 was applied to the frequency of occurrence. The present invention is not limited to a cutoff of 10.

Summary statistics are provided for patient demographics (age and gender), diagnosis group by ICD-10 coding, method of sample collection, frequency of the total number of symptoms and each of the 12 symptoms for the study samples. The overall prevalence and individual prevalence of monomicrobial infection and polymicrobial infection for each of the 24 bacteria were calculated. To differentiate between consortia and random associations of bacteria, a cutoff of 10 patients was applied to the frequency of occurrence. The present invention is not limited to a cutoff of 10.

The measurement for the association between two bacteria is explained by a hypothetical example below.

An example contingency table for two bacteria is shown in Table 5.

TABLE 5

| | | Bacteria 1 | |
|---|---|---|---|
| | | Present | Absent |
| Bacteria 2 | Present | a | b |
| | Absent | c | d |

Phi coefficient (or mean square contingency coefficient) for every pair of the bacteria is calculated as

$$\frac{ad - bc}{\sqrt{( a + b ) ( c + d ) ( a + c ) ( b + d )}}$$

where a, b, c, and d are explained above. The interpretation of Phi coefficient is similar to Pearson correlation coefficient, it ranges from −1 (perfect negative association) to 1 (perfect positive association).

A network diagram was used to visually depict the relationships among the bacteria found most frequently in consortia, and to illustrate the frequency of consortia detected most often and the associated count of symptoms presented in the patients. Lastly, summary statistics and ANOVA comparisons were made for the mean number of symptoms across groups with different numbers of bacteria in consortia. The analyses were performed using SAS version 9.4 (SAS Institute, Inc., Cary, NC) and R 3.5.3

A total of 2493 consecutive patients were enrolled, all 60 years and older. Of the 2493 participants in this analysis, the mean age of patients in this study group was 73.4 years, and 53.9% were female. Voided urine was the most common method of sample collection (2384/2493, 95.6%).

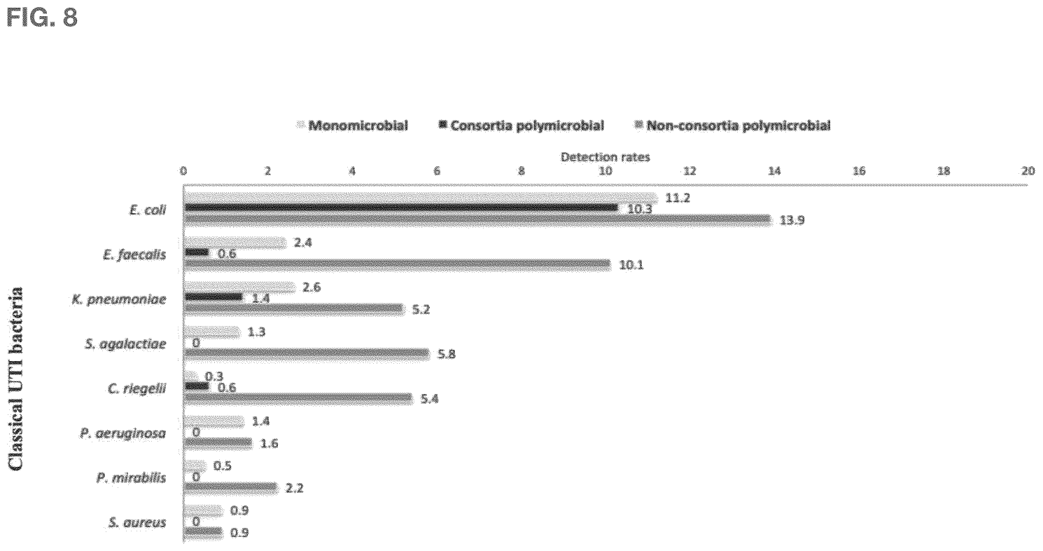

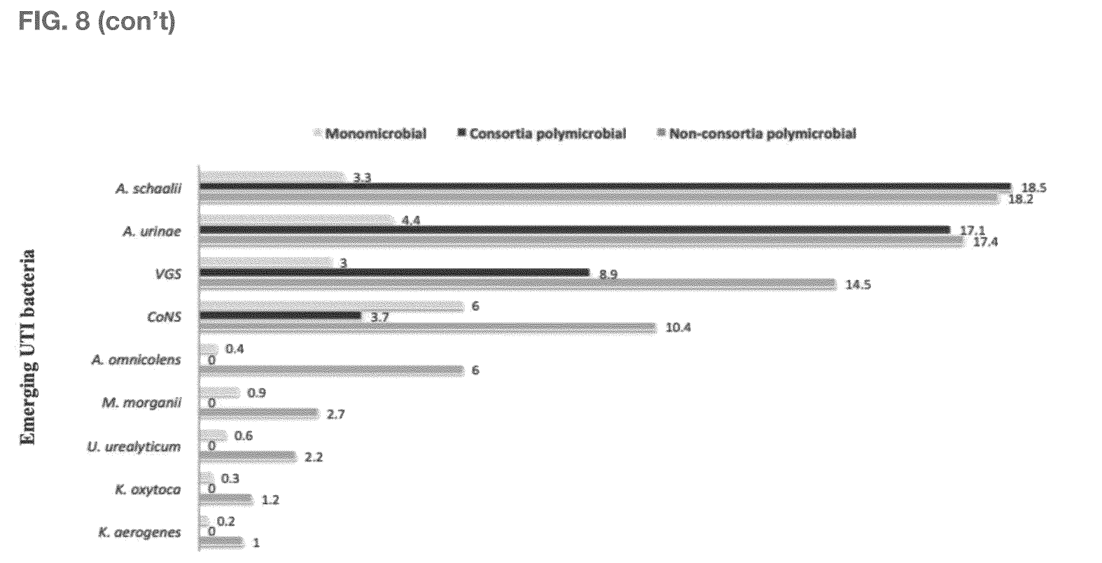

The presence of 24 bacteria was tested in samples provided by 2493 patients from community-based urology practices. M-PCR detected all 24 bacteria in this patient population. FIG. 8 shows the distribution of bacteria detected in the 68.6% (1710/2493) of patients who were positive for bacteria.

In the study population, 31.4% (783/2493) of patients were negative and 68.6% (1710/2493) were positive for bacteria; 27.4% (683/2493) exhibited a monomicrobial UTI. A schaalii (27.4%, 684/2493), A urinae (26.7%, 666/2493), and E coli (24.3%, 605/2493) were the bacteria most frequently detected in this study. These three bacteria were also the most frequently detected bacteria in polymicrobial infections [A schaalii: 61.1% (627/1027), A urinae: 57.5% (591/1027), and E coli: 40.2% (413/1027)] and in consortia [A schaalii-73.0% (316/433), A urinae −62.8% (272/433), and E coli −40.6% (175/433)]. In contrast, the most frequently detected bacteria in monomicrobial infections were E coli (7.7%, 192/2493), Coagulase Negative Staphylococci (4.1%, 102/2493), A urinae (3.0%, 75/2493), A schaalii (2.3%, 57/2493), and Viridans Group Streptococci (2.0%, 51/2493).

Polymicrobial infections were found in 41.2% (1027/2493) of all patients and 60.1% (1027/1710) of patients who tested positive for bacteria. More than 90% of C riegelli (95.4%), A omnicolens (94.4%), and A schaalii (91.7%) were found in polymicrobial infections. Other than M genitalium, which was not found in polymicrobial infections, P aeruginosa (53.8%) and S aureus (51.6%) were the least likely to be detected in polymicrobial infections.

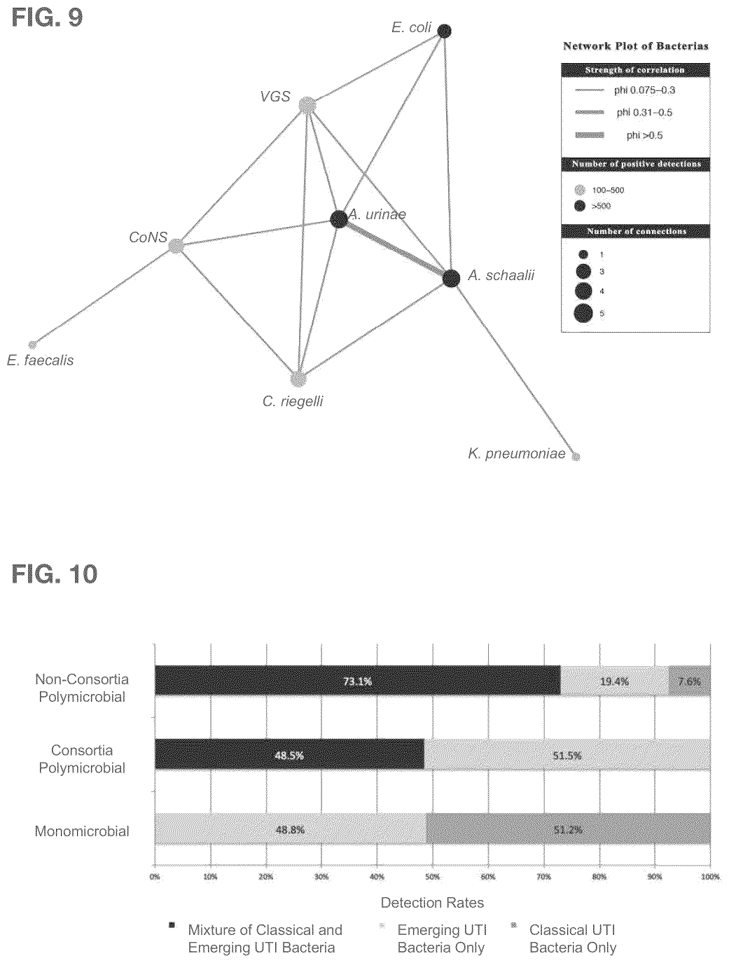

Bacteria in polymicrobial UTIs tended to cluster in groups defined as consortia. Table 6 lists the defined consortia that was detected at the defined cutoff frequency of 10 detections. FIG. 9 shows a network diagram of the relationships among the bacteria found most frequently in consortia. Consortia were found in 17.4% (433/2493) of all patients and 42.2% (433/1027) of polymicrobial infections. Eight bacteria formed 18 different consortia, which ranged in count from 2 to 4 organisms. Eleven of the consortia consisted of two organisms, six consortia contained three organisms, and one consortium involved four organisms.

TABLE 6
Monomicrobial infections, consortia, and clinical findings.

| | | | | | Average number of |
|---|---|---|---|---|---|
| Consortia | Number of | Number of | Bacteria | | clinical findings per |
| ID | bacteria | patients | Classical | Emerging | patient (SD) |
| | All Consortia | 433 | | | |
| | Consortia | 271 | | | 2.8 (1.3) |
| | with 2 | | | | |
| | bacteria | | | | |
| | Emerging | 164 | | | 2.5 |
| | only | | | | |
| A | 2 | 101 | | A. schaalii, | 2.5 |
| | | | | A. urinae | |
| B | 2 | 20 | | A. urinae, VGS | 2.4 |
| C | 2 | 19 | | A. schaalii, | 2.5 |
| | | | | VGS | |
| D | 2 | 14 | | CoNS, VGS | 2.5 |
| E | 2 | 10 | | A. urinae, | 2.5 |
| | | | | CoNS | |
| | Emerging and | 107 | | | 3.2 |
| | Classical | | | | |
| F | 2 | 10 | E. faecalis | CoNS, | 3 |
| G | 2 | 27 | E. coli | VGS | 3.4 |
| H | 2 | 24 | E. coli | A. schaalii | 2.8 |
| I | 2 | 21 | E. coli | A. urinae | 3.4 |
| J | 2 | 15 | E. coli | CoNS | 2.9 |
| K | 2 | 10 | K. pneumoniae | A. schaalii | 3.2 |
| | Consortia | 144 | | | 2.9 (1.1) |
| | with 3 | | | | |
| | bacteria | | | | |
| | Emerging | 59 | | | 2.5 |
| | only | | | | |
| L | 3 | 33 | | A. schaalii, | 2.5 |
| | | | | A. urinae, | |
| | | | | VGS | |
| M | 3 | 15 | | A. schaalii, | 2.5 |
| | | | | A. urinae, | |
| | | | | CoNS | |
| N | 3 | 11 | | A. schaalii, | 2.3 |
| | | | | A. urinae, | |
| | | | | C. riegelii | |
| | Emerging and | 85 | | | 3.1 |
| | Classical | | | | |
| O | 3 | 50 | E. coli | A. schaalii, | 2.9 |
| | | | | A. urinae | |
| P | 3 | 21 | E. coli | A. schaalii, | 3.6 |
| | | | | VGS | |
| Q | 3 | 14 | K. pneumoniae | A. schaalii, | 3.4 |
| | | | | A. urinae | |
| | Consortia | 18 | | | 3.7 (1.4) |
| | with 4 | | | | |
| | bacteria | | | | |
| | Emerging and | 18 | | | 3.7 |
| | Classical | | | | |
| R | 4 | 18 | E. coli | A. schaalii, | 3.7 |
| | | | | A. urinae, | |
| | | | | VGS | |

Clinical findings are a combination of urinalysis results and UTI symptoms.

A schaalii and A urinae constituted the most frequently detected bacteria and the most common combination of bacteria in consortia, being found in 9.7% (242/2493) of all patients, in 38.9% (7/18) of types of consortia, and in 55.9% (242/433) of the instances of consortia. In contrast, some bacteria, such as P aeruginosa and S aureus, were detected in polymicrobial infections but were not found in consortia. Other bacteria, such as C riegelli and E faecalis, appear very often in polymicrobial infections but rarely form consortia. C riegelli exhibited an overall prevalence of 4.4% (109/2493); 95.4% (104/109) of those were found in polymicrobial infections, but only 10.1% (11/109) existed in consortia. Similarly, E faecalis displayed an overall prevalence of 9.0% (224/2493); 81.7% (183/224) of those were found in polymicrobial infections, while only 4.5% (10/224) were in consortia.

There was also a stark contrast in the detection of Gram-positive and Gram-negative bacteria among monomicrobial infections, polymicrobial infections, and consortia; see FIG. 10. Gram negative bacteria constituted 55.1% (376/683) of bacteria detected in monomicrobial infections and 76.4% (2369/3100) of bacteria detected in polymicrobial infections. However, Gram positive bacteria dominated consortia: 80.3% (813/1013) of bacteria detected in consortia were Gram positive. Whereas 41.1% (422/1027) of polymicrobial infections contained only Gram-positive bacteria, 53.8% (233/433) of consortia contained only Gram-positive bacteria. In polymicrobial infections, 56.7% (582/1027) included both Gram positive and Gram-negative bacteria; in consortia, 46.2% (200/433) contained both Gram positive and Gram-negative bacteria. Polymicrobial infections that consisted only of Gram-negative bacteria were rare, being found in 2.2% (23/1027) of infections; there were no consortia that consisted of only Gram-negative bacteria. More than one Gram negative bacterium was detected in 8.7% (89/1027) of polymicrobial infections and in none of the defined consortia. In contrast, more than one Gram positive bacterium was detected in 36.9% (379/1027) of polymicrobial infections and in 77.6% (336/433) of defined consortia.

A relationship between Gram stain classification and symptom presentation also became evident during data analysis. Of the 18 defined consortia, 10 were associated with a clinical presentation of three or more clinical findings. Out of these 10 consortia with 3 or more symptoms, 8 included a Gram-negative bacterium; 6 included E coli, and 2 included K pneumoniae. Another 8 consortia were associated with no more than 2 clinical findings reported during clinical presentation; only one of these included a Gram-negative bacterium, which was E coli. The 8 consortia containing Gram-negative bacteria and associated with both 3 or more symptoms and occurred in 8.4% (209/2493) of all patients and 48.3% (209/433) of patients with consortia. The 8 consortia containing no Gram-negative bacteria and associated with no more than 2 symptoms and occurred in 9.0% (224/2493) of all patients and 51.7% (224/433) of patients with consortia.

This study sought to ascertain the presence of bacterial consortia in patients presenting with symptoms of a UTI from 37 different urology practices across the United States. Using M-PCR, polymicrobial UTIs were detected in 34% of all patients, which is consistent with previous studies. Furthermore, polymicrobial infections were detected in the majority of patients who tested positive for bacteria (60.1%), which is also consistent with previous reports. Defined consortia were detected in 17.4% of all patients and in 42.2% of polymicrobial infections.

In the patient population, 23 different bacteria were detected in polymicrobial infections, but only 8 bacteria were detected in defined consortia. Of these, only four bacteria (A schaalii, A urinae, E coli, and Viridans Group Streptococci) constituted 89.6% of bacteria detected in consortia. Three of these four bacteria (A schaalii, A urinae, and Viridans Group Streptococci) are Gram positive bacteria and formed 75.3% of bacteria detected in consortia. Furthermore, there was no consortium that contained more than one Gram negative bacterium.

A significant shift in the balance of bacteria from predominantly Gram-negative bacteria in monomicrobial and polymicrobial infections to primarily Gram-positive bacteria in consortia was observed in this study. The majority of bacteria in monomicrobial infections (55.1%) and polymicrobial infections (76.4%) were Gram negative. However, consortia consisted of 80.3% Gram positive bacteria. A schaalii, a Gram-positive bacterium, was detected in 77.6% of consortia, but in only 2.3% of monomicrobial infections and 49.0% of non-consortia polymicrobial infections. Similarly, A urinae, another Gram-positive bacterium, was detected in 67.7% of consortia, but in only 3.0% of monomicrobial infections and 50.2% of non-consortia polymicrobial infections. The most common combination of bacteria in both consortia and non-consortia polymicrobial infections was A schaalii and A urinae occurring in 55.9% of consortia but on only 33.0% of non-consortia polymicrobial infections. The balance of bacteria in consortia is shifted not only toward Gram positive bacteria, but toward specific Gram-positive bacteria.

Only two Gram negative bacteria were detected in consortia, E coli and K pneumoniae, occurring in 46.2% of consortia. The shift observed in Gram positive bacteria in consortia was not observed for Gram negative bacteria. E coli occurred in 40.6% of consortia and in 39.9% of non-consortia polymicrobial infections. K pneumoniae was detected in 5.5% of consortia and in 15.0% of non-consortia polymicrobial infections. Further, 77.6% of consortia contained more than one Gram positive bacterium, but no consortium included more than one Gram negative bacterium. In contrast, 68.2% of non-consortia polymicrobial infections included more than one Gram negative bacterium.

This shift in the balance of bacteria in consortia may be interpreted to indicate gain of function dysbiosis. There is significant overgrowth of Gram-positive bacteria, and specifically A schaalii and A urinae. At the same time, there is a shift in detection of Gram-negative bacteria: 13 different Gram-negative bacteria were detected in polymicrobial infections, but only two were detected in consortia.

Although Gram negative bacteria constitute a minority of bacteria in consortia, they appear to be important factors in symptom presentation: patients with consortia containing Gram negative bacteria presented to the clinic with more symptoms than patients with consortia that did not contain Gram negative bacteria. Ten of the 18 types of defined consortia were associated with a larger number of clinical findings, and 8 of those 10 consortia contained Gram negative bacteria. Six of those 8 contained E coli and 2 contained K pneumoniae. Of the 8 consortia associated with fewer clinical findings, only one contained a Gram-negative bacterium, which was E coli.

Microbes form consortia and biofilms because doing so confers growth and survival advantages. A possible contributing factor to these four bacteria forming consortia is that they are not closely related. Previous results have shown that relation at the genus level inhibits the formation of consortia. Genetic diversity within consortia and biofilms is known to increase the overall fitness of the community, producing a more resilient community. Community structures, referred to as urotypes, have been identified in the normal human urobiome. Each normal urotype is dominated by a single genus, with the most common being Lactobacillus, Streptococcus, Gardnerella, and E coli. Urotypes dominated by either A schaalii or A urinae have not been reported. The dominance of UTI consortia by A schaalii and A urinae implies dysbiosis and formation of pathogenic urotypes.

The occurrence of A schaalii or A urinae in 14 of the 18 defined consortia may suggest that they are keystone bacteria in formation of pathogenic consortia. Keystone bacteria often have the capacity to initiate degradation of critical energy substrates in the environment, releasing energy to other members of the consortia. Some have suggested that strategic removal of a keystone bacterium from consortia would cause the consortia to collapse, but evidence indicates that is not likely to be the case. Functional redundancy within consortia may prevent them from collapsing on removal of individual species; complex metabolic networks in bacterial consortia may provide this type of redundancy. Further, functional redundancy may involve other types of networks, including metagenomic, antibiotic resistance and substrate modification.

The findings provide evidence that formation of consortia constitutes dysbiosis in the urobiome. The type of dysbiosis observed is a gain of function, consisting of overgrowth of specific Gram-positive bacteria, A schaalii and A urinae. Gram positive bacteria, in general, are fastidious and not amenable to growth in standard culture. Accumulating evidence that SUC is inadequate because of poor sensitivity has led to an increasing consensus regarding the clinical value of molecular methods for diagnosis of UTI. Recent evidence indicates that A schaalii and A urinae are not detected by culture, but are detected by PCR. The emergence of molecular methods such as PCR as clinical diagnostic tools has been critical for advancing the understanding of polymicrobial UTIs, which tend to be more antibiotic-resistant, and more commonly to lead to urosepsis and increased mortality.

The results show that specific combinations of bacteria in consortia may be more pathogenic and virulent than others.

### Example 3. Bacterial Interactions as Detected by Pooled Antibiotic Susceptibility Testing (P-AST) in Polymicrobial Urine Specimens

The standard of care for diagnosis of urinary tract infections (UTIs) is a standard urine culture (SUC) along with antimicrobial susceptibility testing, and has served to guide treatment since the early 1950s. The methodology relies on an “Escherichia coli (E. coli)-centric” view that perceives UTIs as caused by one or two pathogens. Recent findings, however, reveal that SUC often misses the vast majority of uropathogens and that up to 39% of these infections are polymicrobial in nature. In parallel, antibiotic resistance has been well-studied in monomicrobial infections, but is less characterized in polymicrobial infections. Yet, interactions between bacteria can alter responses to antibiotics.

Currently, antibiotic susceptibility testing (AST) ignores bacterial interactions. In AST, each bacterium is tested in isolation against an antibiotic, providing no opportunity to assess bacterial interactions. Ignoring bacterial interactions can either lead to potential treatment failure or prevent the use of efficacious antibiotics. Both scenarios can have serious clinical consequences.

The present invention discusses the method called Pooled Antibiotic Susceptibility Testing (P-AST), which involves simultaneously growing all detected bacteria together in the presence of antibiotics and then measuring susceptibility. Thus, P-AST considers interactions between cohabiting bacterial species. Urine specimens were obtained from patients presenting with UTI-like symptoms to 37 urology clinics. First, the odds of resistance to 18 antibiotics relative to increasing numbers of bacterial species in a specimen were estimated. We found that antimicrobial susceptibility patterns in polymicrobial specimens differed from those observed in monomicrobial specimens. Since standard of care relies on assessment of antibiotic susceptibility in monomicrobial infections, these findings show that P-AST could serve as a more accurate predictor of antibiotic susceptibility.

This study combines data from two studies of antibiotic resistance patterns in elderly patients presenting with symptoms consistent with a UTI. Retrospective data and patient information (Western IRB number 20171870) were obtained from a single site (Comprehensive Urology, Royal Oak, MI) for 613 patients who presented between March and July 2018. Prospective data and patient information (Western IRB number 20181661) were obtained for 2,511 patients who presented at any of 37 geographically disparate clinics in the United States between July 2018 and February 2019. All subjects met the following inclusion and exclusion criteria. Inclusion criteria included: symptoms of acute cystitis, complicated UTI, persistent UTI, recurrent UTI, prostatitis, pyelonephritis, interstitial cystitis (at any age), symptoms of other conditions at 60 years of age, specimen volumes sufficient to permit urine culture and Multiplex Polymerase Chain Reaction (M-PCR) combined with Pooled Antibiotic Sensitivity Testing (P-AST), patient informed consent, documented times at which the specimens were collected and stabilized with boric acid in grey-top tubes. Exclusion criteria included prior participation in this study, antibiotics taken for any reason other than UTI at the time of enrollment, chronic (≥10 days) indwelling catheters, self-catheterization, and urinary diversion. Antibiotic susceptibility data were available for 1,352 of the 3,124 patients (43.3%).

DNA extraction was performed using the KingFisher/MagMAX™ Automated DNA Extraction instrument and the MagMAX™ DNA Multi-Sample Ultra Kit (ThermoFisher, Carlsbad, CA). 400 μL of urine were transferred to 96-well deep-well plates, sealed, and centrifuged to concentrate the samples, and then the supernatant was removed. Enzyme Lysis Mix (220 μL/well) was added to the samples, which were then incubated for 20 min at 65° C. Proteinase K Mix (PK Mix) was added (50 μL/well) and incubated for 30 min at 65° C. Lysis buffer (125 μL/well) and DNA Binding Bead Mix (40 μL/well) were added, and the samples were vortexed for a minimum of 5 min. Each 96-well plate was loaded into the KingFisher/MagMAX Automated DNA Extraction instrument, which was operated in accordance with standard operating procedures.

DNA analysis was conducted using the Guidance® UTI Test (Pathnostics, Irvine, CA), which consists of both M-PCR and P-AST. Samples were mixed with universal PCR master mix and amplified using TaqMan technology on the Life Technologies 12K Flex OpenArray System™ (Life Technologies, Carlsbad, CA). DNA samples were spotted in duplicate on 112-format OpenArray chips. Plasmids unique to each bacterial species being tested were used as positive controls. Candida tropicalis was used as an inhibition control. A data analysis tool developed by Pathnostics was used to sort data, assess the quality of data, summarize control sample data, identify positive assays, calculate concentrations, and generate draft reports. Probes and primers were used to detect the following pathogenic bacteria: Acinetobacter baumannii, Actinotignum schaalii, Aerococcus urinae, Alloscardovia omnicolens, Citrobacter freundii, Citrobacter koseri, Corynebacterium riegelii, Enterobacter aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Pantoea agglomerans, Proteus mirabilis, Providencia stuartii, Pseudomonas aeruginosa, Serratia marcescens, Staphylococcus aureus, Streptococcus agalactiae, and Ureaplasma urealyticum. Probes and primers also were used to detect the following bacterial groups: Coagulase negative staphylococci (CoNS) (Staphylococcus epidermidis, Staphylococcus haemolyticus, Staphylococcus lugdunensis, Staphylococcus saprophyticus); Viridans group streptococci (VGS) (Streptococcus anginosus, Streptococcus oralis, Streptococcus pasteuranus).

Pooled Antibiotic Susceptibility Testing (P-AST) was performed by aliquoting 1 mL of patient urine specimen into a 1.7 mL microcentrifuge tube. After centrifugation, the supernatant was aspirated and discarded, leaving approximately 500 μL of patient sample in the microcentrifuge tube. One mL of Mueller Hinton Growth Media was then aliquoted into the patient sample in the microcentrifuge tube and the tubes were incubated at 35° C. in a non-CO₂ incubator for 6 hours. Mueller Hinton Agar was used as a negative control. Those samples that reached a minimum threshold of 10,000 cells/mL were then diluted by aliquoting 0.5 mL of sample into a 50 mL conical tube containing Mueller Hinton Growth Media. 96-well plates pre-loaded with antibiotics were then inoculated with diluted samples and incubated along with control plates for 12-16 hours at 35° C. in a single layer. Optical density of samples was then read on a DensiCHEK plate Reader™ (BioMerieux, Marcy-I'Étoile, France).

Logistic regression was used to compare resistance rates in monomicrobial and polymicrobial infections. Specifically, 18 different logistic regression models were fit to the data: the response variable was an indicator of whether the specimen was resistant to the specific antibiotic or not and the predictor variable was an indicator of whether the infection was monomicrobial or polymicrobial. Specimens were classified as monomicrobial if a single bacterial species was detected above the 10000 cells/mL threshold; they were classified as polymicrobial if two or more distinct bacteria species were detected above that threshold. Similar logistic regression models also were run, using the number of distinct bacterial species as the predictor variable.

Interactions between pairs of bacterial species were investigated using a logistic regression model to predict resistance in the presence of specific bacterial species. In this model, the response was an indicator variable of resistance and the predictor variables were 16 indicators of the presence of the following bacterial species or groups that tested positive in at least 30 samples; these were A. schaalii, A. urinae, A. omnicolens, CoNS, C. riegelii, E. faecalis, E. coli, K. oxytoca, K. pneumoniae, M. morganii, P. mirabilis, P. aeruginosa, S. aureus, S. agalactiae, U. urealyticum, and VGS. A backward stepwise model selection was performed on the model with all main effects and all pairwise interactions using an enter significance level of α=0.10 and an exit significance level of α=0.05 to obtain the best fitting model. This model was used to predict resistance rates when a specific bacterial species was present or when a specific pair of species was present.

Using the logistic regression model described above, the resistance rate for a pair of species was compared to the resistance rates for each species alone. Two different principles were applied to calculate the expected resistance rate to a pair of species that do not interact: (a) Highest Single Agent Principle (HSAP) (a commonly used model for drug interactions) and (b) Union Principle (UP) (also used to model drug interactions).

Using the HSAP, a pair of species was considered to have an interaction if the resistance rate of the pair of species was statistically different from the highest resistance rate of each of the two species alone. This model is based on the idea that a pooled specimen containing two species will survive application of a specific antibiotic only if the more resistant species survives. When an antibiotic is applied to the pool, it may kill off species A, but if species B survives, the pool is called resistant.

The UP assumes a pair of bacteria (species A and B) is made up of one genetic variant of species A and one genetic variant of species B, and that the pool is resistant if either species A is resistant or if species B is resistant. If species A is resistant with probability P(A), and species B is resistant with probability P(B), then the probability of resistance of the pool is:

P(pool resistance)=P(A)+P(B)−P(A)P(B)

This assumes the two species do not interact, and therefore act independently.

Interactions were statistically tested using bootstrapped samples of the 1,352 patients with antibiotic resistance results; each patient was randomly selected with replacement. A logistic regression with terms in the best fitting model selected as above was fit to each bootstrapped sample. The predicted resistance when the pair of species was present was compared to the predicted resistance to each species alone using the model fit to the bootstrap sample (assuming either the HSAP or UP). 5000 bootstrapped samples were generated and analyzed. If 97.5% or greater of the bootstrapped samples demonstrated a pool resistance higher than expected, the interaction was deemed to show a statistically significant interaction with increased resistance. If 97.5% or greater of the bootstrapped samples demonstrated a pool resistance lower than expected, the interaction was deemed to show a statistically significant interaction with decreased resistance.

A total of 3,124 patients, from two studies and from 37 geographically disparate urology clinics in the United States, presenting with symptoms consistent with a UTI, were initially included in the study. P-AST data were available for 43.3% (1352) of these patients. The demographics are as follows: the mean age was years, 66% percent (887/1,352) were female, whereas 34% (465/1,352) male.

By M-PCR, 38.9% (1,214/3,124) of the specimens were negative for bacteria, whereas 61.1% (1910/3124) were positive. P-AST data were available for 1,352 (70.7%) of these 1,910 positive specimens. Of these 1,352 specimens, 43.9% (594/1352) were monomicrobial, whereas 56.1% (758/1352) were polymicrobial.

Five hundred and fifty eight positive samples lacked antimicrobial susceptibility data for the following reasons: (1) no species were detected at 10,000 cells/mL (correlating to colony forming units/mL) and thus no species were tested against antibiotics; (2) the species detected by PCR were fastidious (i.e., they required specific growth conditions, extremely restrictive growth conditions, or extreme length of time in order to perform susceptibility testing); (3) prior antimicrobial use caused bacteria to fail to thrive in the P-AST assay; or (4) species were not identified because the M-PCR reaction was inhibited (based upon comparison to negative and positive controls). M-PCR inhibition can occur when an interfering substance prevents the amplification and subsequent detection of the PCR product associated with targeted DNA.

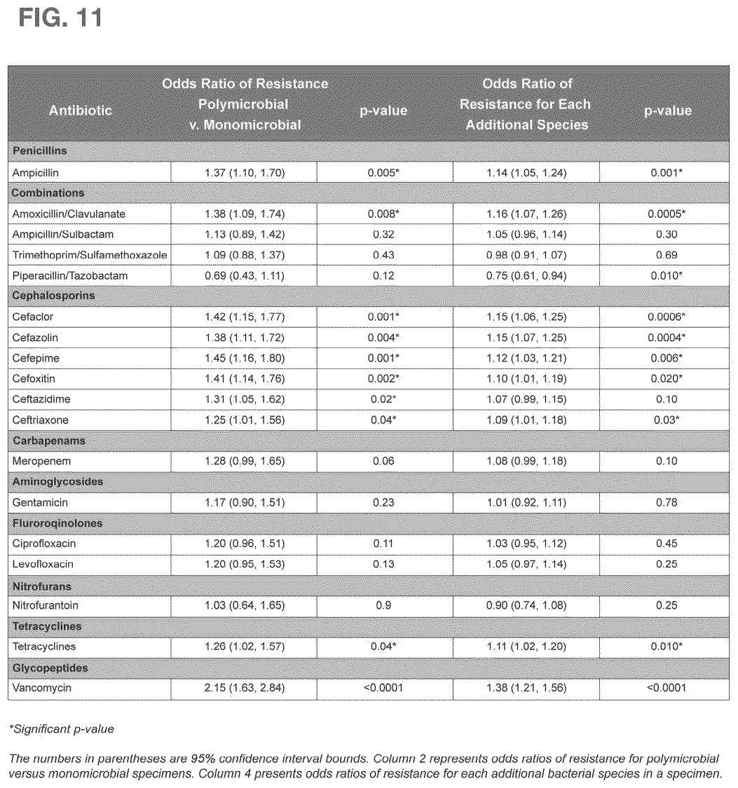

Odds ratios of antibiotic resistance in polymicrobial versus monomicrobial specimens are shown in FIG. 11, along with the odds ratio of resistance for each increase in the number of bacterial species in polymicrobial specimens. The resistance rates of polymicrobial samples were generally higher than the rates of monomicrobial samples; 10 of 18 antibiotics had statistically higher resistance rates for polymicrobial samples. The odds of resistance for each additional species identified in a polymicrobial specimen increased for ampicillin, amoxicillin/clavulanate, five of the six of the cephalosporins tested, vancomycin, and tetracycline. The opposite was true for piperacillin/tazobactam, where each additional species in a polymicrobial specimen resulted in a 75% decrease in the odds of resistance (95% CI 0.61, 0.94, p=0.01).

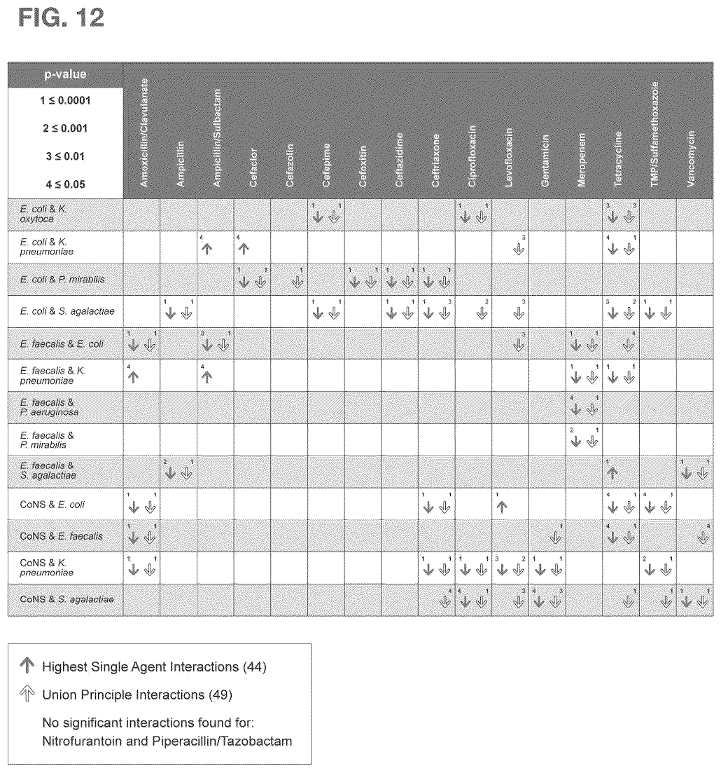

FIG. 12 shows the effect of specific species interactions on the probability of increased or decreased resistance to each antibiotic tested. No interactions were detected for nitrofurantoin and piperacillin/tazobactam. Whereas the odds of resistance to ampicillin, amoxicillin/clavulanate, 6 different cephalosporins, vancomycin, and tetracycline increased with increasing number of detected species, there were 19 instances for which 11 of the 13 bacterial pairs resulted in reduced susceptibility to the same antibiotics.

Using HSAP, there were 44 instances for which 13 pairs of bacteria showed statistically significant interactions that either increased or decreased the probability of resistance to the antibiotics tested. According to the HSAP principle, most interactions resulted in a decreased probability of resistance. Only 6/44 (13.6%) pairings resulted in increased odds of antibiotic resistance, whereas a decreased probability occurred in 38/44 (86.4%) of pairings.

The bacterial combinations that increased the probability of antibiotic resistance according to the HSAP model were E. faecalis and K. pneumoniae (amoxicillin/clavulanate, p=0.02 and ampicillin/sulbactam, p=0.03), E. coli and K. pneumoniae (ampicillin/sulbactam, p=0.04 and cefaclor, p=0.05), CoNS and E. coli and (levofloxacin, p<0.001), E. faecalis and S. agalactiae (tetracycline, p<0.001).

The UP model identified 49 statistically significant interactions, all of which showed decreased probability of resistance to the antibiotics tested.

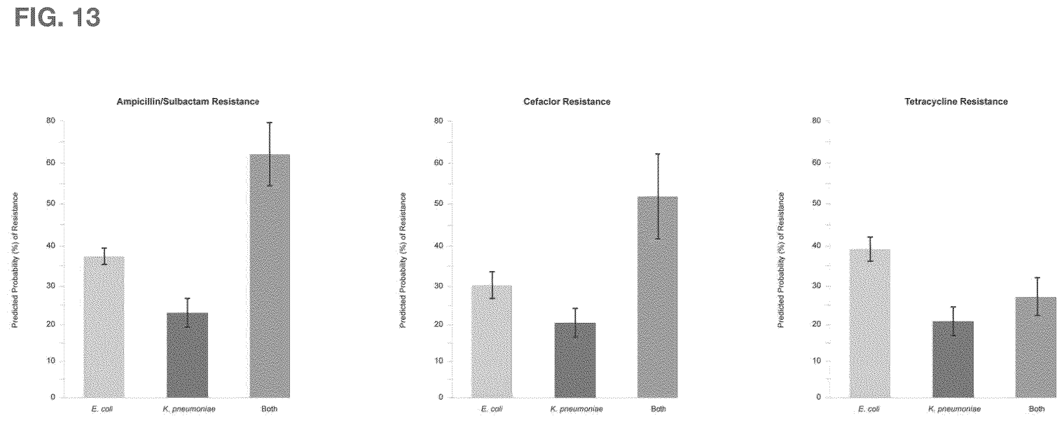

To illustrate the model, one specific pair is presented in graphical form. FIG. 13 shows the predicted probabilities of resistance to ampicillin/sulbactam, cefaclor, and tetracycline by monomicrobial positive cultures for E. coli and K. pneumoniae and a polymicrobial culture positive for both E. coli and K. pneumoniae. When the HSAP model was used, the pairing of E. coli and K. pneumoniae resulted in either a significant increase or significant decrease in the probability of resistance depending on the antibiotic tested. For example, when ampicillin/sulbactam or cefaclor was applied to the combination of E. coli and K. pneumoniae, the resistance rate was higher than either E. coli or K. pneumoniae alone. In contrast, the resistance rate to tetracycline of same combination of species, E. coli and K. pneumoniae, was intermediate between the resistance rates to each species alone.

These results demonstrate that polymicrobial infections, which constituted 56.1% (758/1,352) of positive samples with susceptibility results, can alter response to antibiotics. They also show that the alteration is sensitive to both the specific bacterial combination and the antibiotic tested. Thirteen bacterial pairs had one or more significant interactions when tested on 16 of the 18 antibiotics using HSAP and UP. Of these interactions, 38 resulted in a decreased probability of resistance, while 6 resulted in an increased probability of resistance. The combination of E. coli and K. pneumoniae resulted in an increased probability of resistance to ampicillin/sulbactam and cefaclor, but decreased probability of resistance to tetracycline. E. faecalis together with K. pneumoniae resulted in increased resistance to amoxicillin/clavulanate and ampicillin/sulbactam, but decreased resistance to levofloxacin, meropenem, and tetracycline. E. faecalis combined with S. agalactiae produced an increase in resistance to tetracycline, but decreased resistance to ampicillin and vancomycin. Similarly, the combination of CoNS and E. coli produced an increased probability in resistance to levofloxacin, but the same combination produced a decreased probability in resistance to amoxicillin/clavulanate, ceftriaxone, tetracycline, and trimethoprim/sulfamethoxazole. These differences may be attributed to the unique mechanisms of action of the specific antibiotics.

A similar set of contrasts is observed from the perspective of individual antibiotics. Different pairs of bacteria caused both increased and decreased resistance to amoxicillin/clavulanate, ampicillin/sulbactam, cefaclor, levofloxacin, and tetracycline. For instance, E. coli combined with K. pneumoniae produced an increase in resistance to cefaclor, while E. coli combined with P. mirabilis produced a decrease in resistance to cefaclor. These results highlight the importance of accurate identification of bacteria in polymicrobial infections: a difference in identification of one species can influence antibiotic resistance.

The observed effects on antibiotic resistance in polymicrobial infections may be due to cooperative and/or competitive interactions between bacteria. Resistant bacteria can cooperatively protect susceptible bacteria by degrading antibiotics, as occurs when secreted beta-lactamase degrades beta-lactam antibiotics. Antibiotic resistance can be conferred by one bacterium on another bacterium by means of horizontal gene transfer (HGT) of antibiotic resistance genes. Bacterial interactions with host macrophages can promote HGT. For instance, P. aeruginosa, when present in biofilms, produces extracellular DNA that induces neutrophils to produce pro-inflammatory cytokines (IL-8 and IL-1 beta). The ensuing inflammation can promote HGT involving E. coli. Interestingly, some antibiotics can also promote HGT: antibiotics that cause bacterial lysis release DNA and proteins that can be taken up by other bacteria. In addition, one bacterium can stimulate gene expression in another bacterium, resulting in upregulation of efflux pumps leading to increased antibiotic resistance. Bacterial community spatial structuring within a polymicrobial biofilm may also affect the efficacy of antibiotics.

Decreased resistance to antibiotics in polymicrobial specimens may also be due to competitive mechanisms between bacteria. P. aeruginosa has been documented to produce antibiotics, whereas Enterococcus species produce and secrete bacteriocins. Gram-negative bacteria have developed a number of specialized secretion systems that can perform protective functions. Type V secretion systems secrete proteases that digest IgA, surface receptors that bind the constant region of IgG, and virulence factor/adhesin proteins that promote colonization. Type VI secretion systems allow Gram-negative bacteria to secrete antibacterial toxins directly into other bacteria. At the same time, Type VI systems mediate DNA acquisition via HGT; an example is the capacity for A. baumannii to rapidly acquire resistance genes from E. coli by means of Type VI transfer systems.

One type of bacterial interaction can cause a paradoxical result: cross-feeding between bacteria can produce decreased antibiotic resistance. This may explain our observed decreased probability of antibiotic resistance seen in most specific organism combinations. Cross-feeding is a process by which one organism produces metabolites that promote the survival of another organism. However, this interaction can produce a chain of dependencies, leaving the entire chain only as resistant as the most susceptible bacterium. Adamowicz et al. showed that bacterial species were inhibited at significantly lower antibiotic concentrations in cross-feeding communities than in monoculture, coined as the “weakest link” model.

Strengths of the study include the large number of samples coming from multiple urology clinics across the United States. Additionally, interactions were analyzed using specimens collected from the clinical setting. The specific interactions impacting the observed susceptibility patterns need to be examined.

Bacteria are social organisms that interact within and between species. Key interactions play critical roles in the growth, pathogenesis, and virulence of bacterial species. Because of these key and specific interactions, correct identification of bacterial species increases in significance. In order to accurately assess these interactions and provide clinical context, we have developed P-AST, a pooled antibiotic susceptibility test. Using this methodology, we observed both increased and decreased antibiotic susceptibility based on the type of species observed, as well as the class of antibiotic administered. Elucidation of the molecular mechanisms by which alterations in antibiotic response occur in polymicrobial infections will require additional research. Based on these findings, P-AST testing might more closely approximate the polymicrobial environment in the patient and possibly provide more clinically important information regarding antibiotic susceptibility.

### Example 4. Utilization of M-PCR and P-AST for Diagnosis and Management of UTIs in Home-Based Primary Care

The present invention describes utilization of M-PCR in addition to P-AST to determine antibiotic susceptibility and resistance of the organisms identified by M-PCR. Antibiotic susceptibility assays that test individual pathogens against antibiotics may miss interactions between bacteria in polymicrobial environments. The P-AST tests all pathogens within a urine specimen simultaneously against antibiotics and can be informative about changes in antibiotic response due to bacterial interactions. Inventors have recently shown that bacterial interactions in polymicrobial infections could alter antibiotic susceptibilities. These changes can significantly affect the type and dosage of antibiotics required to treat the patient. Further, the use of the M-PCR/P-AST test significantly reduces turnaround time, providing results in 24 hours, with SUC taking up to 5 days to result. Thus, concurrently, the M-PCR/P-AST assay quickly detects 2 organisms or more while also providing susceptibility information in polymicrobial samples. In the present retrospective study, it was evaluated whether M-PCR/P-AST was superior to SUC using ED utilization and hospitalization rates as proxy measures of outpatient treatment effectiveness: more effective outpatient treatment should result in lower rates for ED utilization and hospitalization.

This was an observational, retrospective study that compared the number of ED visits and hospital admissions between two patient cohorts. The study population consisted of patients seen by primary care providers who attend to patients in their homes or assisted living facilities. All patients in this study were seen for possible UTI. Patients were divided into two time period cohorts. The SUC cohort consisted of patients who received outpatient treatment with antibiotics based upon results from SUC between Mar. 1, 2016 and Jul. 31, 2017. There was a washout period of 7 months (Aug. 1, 2017-Feb. 28, 2018). The M-PCR/P-AST cohort included patients who received outpatient treatment with antibiotics based upon results from M-PCR testing between Mar. 1, 2018 and Jul. 31, 2019. Urine samples were obtained from patients by either self-administered clean catch or catheterization. Samples were collected and transported to Lab Corp (Various Locations in US) or Quest Diagnostics (Various Locations in US) for testing by culture using standard procedures.

DNA extraction was performed using the KingFisher/MagMAX™ Automated DNA Extraction instrument and the MagMAX™ DNA Multi-Sample Ultra Kit (ThermoFisher, Carlsbad, CA). 400 μL of urine were transferred to 96-well deep-well plates, sealed, and centrifuged to concentrate the samples, and then the supernatant was removed. Enzyme Lysis Mix (220 μL/well) was added to the samples, which were then incubated for 20 min at 65° C. Proteinase K Mix (PK Mix) was added (50 μL/well) and incubated for 30 min at 65° C. Lysis buffer (125 μL/well) and DNA Binding Bead Mix (40 μL/well) were added, and the samples were vortexed for a minimum of 5 min. Each 96-well plate was loaded into the KingFisher/MagMAX Automated DNA Extraction instrument, which was operated in accordance with standard operating procedures.

DNA analysis was conducted using the Guidance® UTI Test (Pathnostics, Irvine, CA), which consists of both M-PCR and P-AST. Samples were mixed with universal PCR master mix and amplified using TaqMan technology on the Life Technologies 12K Flex OpenArray System™ (Life Technologies, Carlsbad, CA). DNA samples were spotted in duplicate on 112-format OpenArray chips. Plasmids unique to each bacterial species being tested were used as positive controls. Candida tropicalis was used as an inhibition control. A data analysis tool developed by Pathnostics was used to sort data, assess the quality of data, summarize control sample data, identify positive assays, calculate concentrations, and generate draft reports. Probes and primers were used to detect the following pathogenic bacteria: Acinetobacter baumannii, Actinotignum schaalii, Aerococcus urinae, Alloscardovia omnicolens, Citrobacter freundii, Citrobacter koseri, Corynebacterium riegelii, Enterobacter aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Pantoea agglomerans, Proteus mirabilis, Providencia stuartii, Pseudomonas aeruginosa, Serratia marcescens, Staphylococcus aureus, Streptococcus agalactiae, and Ureaplasma urealyticum. Probes and primers also were used to detect the following bacterial groups: Coagulase negative staphylococci (CoNS) (Staphylococcus epidermidis, Staphylococcus haemolyticus, Staphylococcus lugdunensis, Staphylococcus saprophyticus); Viridans group streptococci (VGS) (Streptococcus anginosus, Streptococcus oralis, Streptococcus pasteuranus). Reporting included both the name of the organism identified and semi quantified counts of organisms. Counts were reported in cells/mL and correlated to Colony Forming Units (CFU).

P-AST was performed by aliquoting 1 mL of patient urine specimen into a 1.7 mL microcentrifuge tube. After centrifugation, the supernatant was aspirated and discarded, leaving approximately 500 μL of patient sample in the microcentrifuge tube. One mL of Mueller Hinton Growth Media was then aliquoted into the patient sample in the microcentrifuge tube and the tubes were incubated at 35° C. in a non-CO₂ incubator for 6 hours. Mueller Hinton Agar was used as a negative control. Those samples that reached a minimum threshold of 10,000 cells/mL were then diluted by aliquoting 0.5 mL of sample into a 50 mL conical tube containing Mueller Hinton Growth Media. 96-well plates pre-loaded with antibiotics were then inoculated with diluted samples and incubated along with control plates for 12-16 hours at 35° C. in a single layer. Optical density of samples was then read on a DensiCHEK plate Reader™ (BioMerieux, Marcy-I'Étoile, France).

Patient demographic, comorbidity, and facility-level data were collected for both groups of patients using the Health Catalyst (data warehouse). Demographic variables included age and gender. Additionally, the Charlson/Deyo (CD) Index Score was collected along with the number of per-patient physician visits.

ED visit and hospital admission rate for UTI were identified using the primary diagnosis code from files available through the Centers for Medicare and Medicaid Services (CMS). Study data included medical records with both ICD-9 and ICD-10 codes. ICD-9 codes were still being used during the time period for the SUC cohort until the transition to ICD-10 coding took effect during the time period for the M-PCR/P-AST cohort. Applicable ICD-9 diagnosis codes include 5990 (Urinary tract infection), 788.1 (Dysuria), 590.10 (Acute pyelonephritis), 590.80 (Pyelonephritis), 5999 (Urinary tract disease), and 59000 (Chronic pyelonephritis). ICD-10 diagnosis codes include N39.0 (Urinary tract infection, site not specified), R30.0 (Dysuria), R35.0 (Frequency of micturition), R32 (Unspecified urinary incontinence), Z87.440 (Personal history of urinary (tract) infections). Cases were excluded if records indicated that the National Provider Identifier (NPI) did not match that of a listed investigator participating in the study, if a patient resided in hospice, or if the diagnosis code was missing.

Differences between the cohort 1 (SUC) and cohort 2 (M-PCR/P-AST) cohorts were analyzed using standardized difference (SD). The SD was calculated as the difference in means (or proportions) divided by the pooled estimate of the standard deviation, with the value of 0.1 considered negligible difference and 0.2 to considered a small difference between the two groups. SD is increasingly used to compare balances in baseline covariates between study groups because unlike other statistical tests, such as Student's t-test that produces p values; the SD is not influenced by the sample size. At the same time, it also allows for the comparison of the variables measured in different units.

The average number of ED visits and/or hospitalizations for UTI for each cohort was calculated. A generalized linear model was used to compare the number of ED visits and/or hospitalizations between the two cohorts, using the negative binomial distribution with log link to account for over-dispersion of the count data. The dependent variable was the count data for the number of ED visits and/or hospitalizations per patient per cohort, and the independent variable was the cohort. Multiple UTI events per patient could confound the number of ED visits and/or hospitalizations. Also conducted was a patient-level analysis by summarizing the frequencies and the proportion of patients with any ED or hospitalization event for UTI. A logistic regression model with logit link was used for this binary outcome to assess the difference between the two groups. The dependent variable was the dichotomized variable of any hospitalization and/or ER visit per patient per cohort; the independent variable was the cohort.

The per-patient frequencies of hospitalization/ED visits by the two cohorts was described in order to elucidate the number of patients who experienced multiple events. Among those patients with any ED or hospitalization visit, the gender distribution by cohort was assessed. The analyses were performed using SAS version 9.4 (SAS Institute, Inc., Cary, NC) and StataSE 16.

Overall, the retrospective chart review included 34,414 unique patients in the SUC cohort and 31,967 unique patients in the M-PCR/P-AST cohort.

The two cohorts were similar, as the SD for demographic variables were less than 0.20 for all variables. The mean age for the SUC cohort was 73.5 years and 72.9 years for the M-PCR/P-AST cohort. There was a negligible difference in the number of patients seen in Ohio, Florida, and Washington. Fewer patients were seen in Ohio for the M-PCR/P-AST cohort (23% v 17.1%, SD=−0.148) while more patients were seen in Washington (1.5% v 3.4%, SD=−0.097) and Florida (8.3% v 11.7%, SD=0.112). The gender breakdown was similar in the two cohorts, with 62.9% of patients being female in the SUC cohort and 62% female in the M-PCR/P-AST cohort. The mean number of physician visits was 6.2 for the SUC cohort and 6.1 for the M-PCR/P-AST cohort, with a median of 5.0 for both cohorts.

Compared to patients in the SUC cohort, patients in the M-PCR/P-AST cohort visited the ED and/or were admitted to the hospital less frequently. Of the 34,414 patient cases in the SUC cohort, there were a total of 1,307 (3.79%) events of either ED visits or hospitalizations during the study period. By contrast, 1,045 (3.27%) similar events took place in the 31,967 patient cases reviewed in the M-PCR/P-AST cohort, which translated to a statistically significant (p=0.003) difference between the two cohorts.

Overall, the number of ED visits decreased between the two cohorts, from 802 (2.33%) for the SUC cohort to 657 (2.06%) for the M-PCR/P-AST cohort, but this difference bordered on statistical significance (p=0.047). The number of hospitalizations was significantly lower in the M-PCR/P-AST cohort (388; 1.21%) than in the SUC cohort (509, 1.48%; p=0.008). Multiple hospitalizations per patient could confound the number of hospitalizations. If each patient was counted only once per UTI event, there was a statistically significant reduction in patients either utilizing the ED or being hospitalized, from 1,041 (3.02%) for the SUC cohort to 831 (2.60%, p=0.001) for the M-PCR/P-AST cohort.

Regarding the patient-level distribution of ED or hospitalization frequencies, one patient could experience multiple ED or hospital utilization events over the study time-period, with a maximum of 9 events per patient. Slightly fewer patients in the M-PCR/P-AST cohort (150, 0.47%) experienced multiple events than the SUC cohort (180, 0.52%; p=0.32).

The data was analyzed to determine if there were relationships between age and number of ED visits and/or hospitalizations for those patients with multiple events. Overall, those patients not experiencing an ED/hospitalization event, or experiencing 1-3 events, were similar in age. A trend is seen that patients in the M-PCR/P-AST cohort who experienced 4 or more visits were older than patients in the SUC cohort, yet the results show that the ED visit and hospitalization rates for the M-PCR/P-AST cohort were smaller than the SUC cohort.

This study attempts to assess the impact transitioning testing for patients, from SUC to M-PCR/P-AST, had on ED and hospitalization rates. VPA had moved away from SUC based on reports in the literature suggesting that SUC had a >30% false-negative rate and missed significant numbers of uropathogens. An additional motivation in internalizing this test was to reduce the turn-around associated with SUC testing to decrease empiric treatment. The study sample was divided into two cohorts, one in which UTI treatment decisions were made based upon results from SUC and antimicrobial susceptibility testing, and the other in which UTI treatment decisions were made based upon results from M-PCR/P-AST. The two cohort populations were similar in age, gender, race, CD Risk Score, location, and the number of provider visits.

It was found that a significant association between the use of the M-PCR/P-AST assay and a reduction in ED utilization and hospital admission rates. Specifically, ED utilization and hospital admission rates for UTI were higher in patients for whom prior outpatient treatment was guided by results from SUC than patients for whom prior outpatient treatment was guided by results from the M-PCR/P-AST assay. This association was stronger for hospitalization rates than for ED utilization. Further, there were fewer repeated hospitalization events for patients managed based upon results from the M-PCR/P-AST assay (0.47%, 150/31,967) than for patients managed using SUC (0.52%, 180/34,414, p=0.32).

This study compared an assay that combines M-PCR detection of pathogens and P-AST with traditional urine culture in the context of UTI treatment. We found a statistically significant association between the use of M-PCR/P-AST and reductions in both ED use and hospitalizations. Greater diagnostic accuracy and shorter turn-around time provided by M-PCR/P-AST may have contributed to this finding.

### Example 5: Antibiotic Resistance (ABR) Testing Plates

Referring to other disclosed methods herein, samples may be collected from subjects according to standard collection protocols in sterile containers and are transported to the testing facility.

An example of the preparation of the antibiotic resistance (ABR) testing plates involves two steps. First is preparation of antibiotic solutions and the second is preparation of the bacterial growth medium plate. The antibiotics to be tested for any given sample include antibiotics known to be useful for treating the tissue having the suspected infection, or any antibiotics requested by a medical or laboratory professional having knowledge of the particular patient sample. It is anticipated that most assays will be performed with a standard panel of antibiotics based on the type and location of infection suspected by a medical professional. In some embodiments, the standard panel of antibiotics comprises one or a combination of nitrofurantoin, ciprofloxacin, meropenem, ceftriaxone, trimethoprim/sulfamethoxazole, piperacillin/tazobactam, levofloxacin, cefoxitin, tetracycline, ampicillin/sulbactam, ampicillin, tetracyline, celfaclor, cefazolin, amoxicillin/clavulant, ceftazidime, fosfomycin, cefuroxime, cephalalexine, vancomycin, any other antibiotic disclosed herein including the figures, the like, or a combination thereof. However, patients with known antibiotic allergies or sensitivities, or with a history of antibiotic resistance, may require customized panels of antibiotics. The assay can be performed simultaneous with an unlimited number of antibiotics.

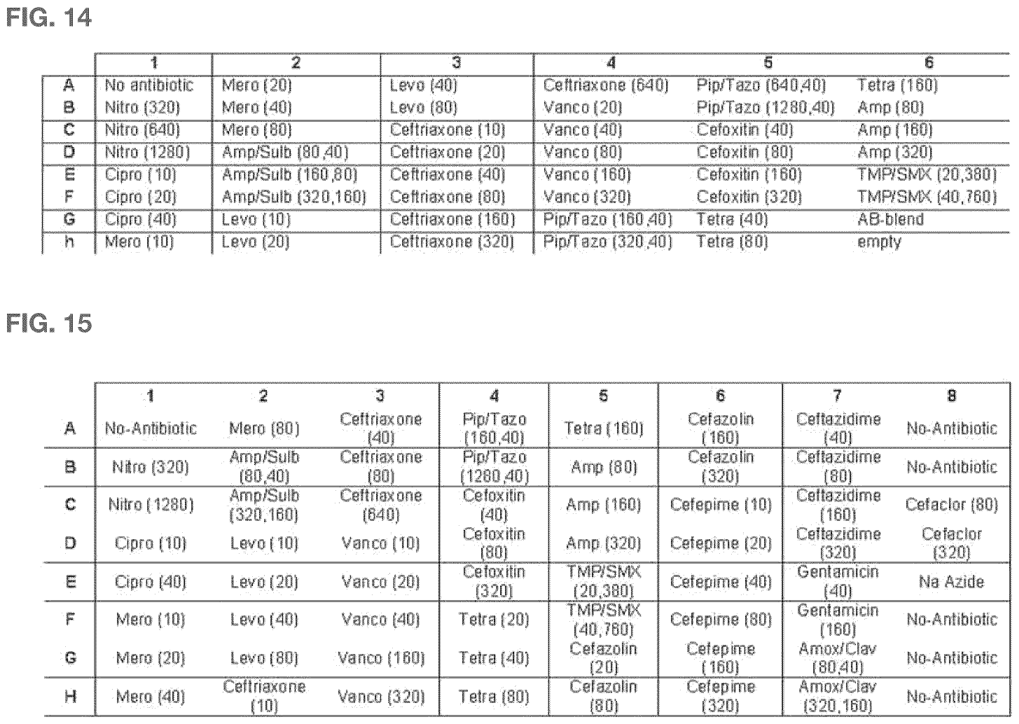

Antibiotic stock solutions are prepared using solvents suitable for each antibiotic and then 10× solutions are prepared and stored in multi-well plates to allow efficient transfer to testing plates. Each antibiotic is tested at a minimum of concentrations. In some embodiments, three concentrations, four concentrations, five concentrations, six concentrations, seven concentrations, eight concentrations, nine concentrations, or ten concentrations of an antibiotic, or antibiotic combination, are included in the assay. Typically serial dilutions of the antibiotics are prepared wherein each dilution represents half the concentration of the higher concentration. The 10× antibiotic solutions are stored in the multi-well plate according to a plate plan established for the antibiotic panel chosen for the assay. Exemplary plate plans are depicted in the Antibiotic Source Plates in FIG. 14 and FIG. 15. Antibiotic stocks and 10× solutions are stored at 2-8° C. until needed.

The ABR testing plates may be multi-well plates (e.g., 6-well, 12-well, 24-well, 48-well, 96-well, 384-well plates, or any multi-well plate suitable for this purpose) capable of containing bacterial growth medium and culturing bacteria. In some embodiments, the plates are 96-well plates. In some embodiments, sterile agar-bacterial growth medium is dispensed into each well of the plate. Exemplary agar-bacterial growth medium include, but are not limited to Mueller-Hinton agar, blood agar, trypticase soy agar, etc. After the agar has solidified at room temperature, 1/10 volume (of bacterial growth medium) of 10× antibiotic solution is added to each well of the test plate according to the predetermined plate plan. After the antibiotics have been introduced to the bacterial growth medium, the plates are allowed to rest for at least one hour. For long-term storage, the antibiotic-containing ABR plates are stored at 2-8° C. In some embodiments, sterile liquid broth bacterial growth medium mixed with sample is dispensed into each well of the plate containing 1/10 volume (of bacterial growth medium) of 10× antibiotic solution arrayed according to a predetermined plate plan. Multi-well plates containing 1/10 volume (of final well volume of bacterial growth medium and antibiotic solution) are stored at 2-8° C. for later use or long-term storage.

Samples for the disclosed antibiotic resistance testing may be optionally diluted in sterile aqueous solution or mixed with bacterial growth medium. In some embodiments, a volume of sample for the disclosed antibiotic resistance testing are first mixed with a growth medium and incubated for 0-24 hours at an incubation temperature of 35±4° C. The samples are then diluted with saline and then mixed with growth medium and added to room temperature ABR testing plates at 9/10 volume of each well in the multi-well plate. In some embodiments, samples are added to room temperature ABR plates at 1/20 volume of bacterial growth medium present in the well. A single patient specimen is used for each ABR plate. If multiple patient specimens are to be tested, each specimen is assayed in its own plate. Once inoculated, the plates are covered and incubated to encourage bacterial growth. Embodiments where a single sample is assayed using more than one plate are also within the scope of the present method.

The plates can be used to culture either anaerobic or aerobic bacteria. For culture of anaerobic bacteria, the plates are incubated at a temperature and in a reduced-oxygen environment to encourage growth of anaerobic bacteria. For culture of aerobic bacteria, the plates are incubated at a temperature and in an oxygen-containing environment to encourage growth of aerobic bacteria.

The incubation temperature can vary depending on the expected types of bacteria but will most likely be in a range of 35-40° C. The plates containing samples are incubated for 12-48 hours, 12-24 hours, 24-28 hours, 12-36 hours, 14-30 hours, 16-24 hours, 16-20 hours, or 16-18 hours, or any range bounded by these numbers.

In some embodiments, wherein the assay is performed with an agar-containing medium, after incubation, bacteria present in each well are recovered by resuspension in an aqueous liquid. Suitable liquids include, but are not limited to, water, saline, culture medium, etc. The aqueous liquid should be sterile, or at least free from bacterial growth. A volume of liquid equal to 100% of the volume of bacterial growth medium is carefully added to the wells of the ABR plate and allowed to sit for at least 30 minutes. In some embodiments, the plates are allowed to sit for 35 minutes, 40 minutes, 45 minutes, 50 minutes, or 60 minutes. The resulting suspension is then carefully removed from each well into individual wells of a clean multi-well plate according to the predetermined plate plan. The plates are optionally agitated to cause mixing of the bacteria with the liquid prior to removal of the suspension. In some embodiments wherein the assay is performed using liquid growth medium, the multi-well plate will be applied to OD₆₀₀ measurement immediately after incubation.

The multi-well plate containing the bacteria-containing suspension is then read in a spectrophotometer. The optical density of the recovered liquid is measured at OD₆₀₀ multiple times to correct for uneven distribution of bacteria particles in the suspension. In some embodiments, the plates are read one time, two times, three times, four times, five times, six times, seven times, or eight times. The multiple plate reads occur in sequence without allowing the suspension to settle in the wells.

The multiple OD₆₀₀ of each well are averaged to provide an accurate quantitation of bacteria present in each well under the specific conditions. Each well's average OD₆₀₀ is then adjusted for background by subtracting the average OD₆₀₀ measurements of a well where no bacteria could grow to yield a blanked value. In some embodiments, this no-growth well contains a blend of antibiotics (AB-blend). In some embodiments, this no-growth well contains sodium azide (Na-Azide). The blanked value is representative of the ability of bacteria to grow in the presence of the particular antibiotic in the well.

The blanked results are then converted into a “resistance” (R) or “sensitive” (S) score based on a threshold value. OD₆₀₀ measurements greater than or equal to the threshold are interpreted as resistant, while measurements below the threshold are interpreted as sensitive.

In some embodiments, the threshold value is for an agar-containing medium. In some embodiments, a threshold value has been determined at 0.010 to 1.000, 0.010-0.090, 0.015 to 0.035, or 0.020 to 0.030 based on correlations to a standard reference method. In some embodiments, the threshold value as been determined at about 0.010, about 0.015, about 0.020, about 0.025, about 0.030, about 0.035, about 0.040, about 0.045, about 0.050, about 0.055, about 0.060, about 0.065, about 0.070, about 0.075, about 0.080, about 0.085, or about 0.090 based on correlations to a standard reference method. In some embodiments, a threshold value has been determined at 0.025 based on correlations to a standard reference method.

In some embodiments, the threshold value is for a liquid medium. In some embodiments, a threshold value has been determined at 0.010-1.000, 0.020-0.090, 0.050-0.080, 0.055 to 0.075, or 0.060 to 0.070 based on correlation to a consensus score between two standard reference methods. In some embodiments, the threshold value as been determined at about 0.010, about 0.015, about 0.020, about 0.025, about 0.030, about 0.035, about 0.040, about 0.045, about 0.050, about 0.055, about 0.060, about 0.065, about 0.070, about 0.075, about 0.080, about 0.085, about 0.090, or about 0.095 based on correlation to a consensus score between two standard reference methods. In some embodiments, a threshold value has been determined at 0.065 based on correlation to a consensus score between two standard reference methods.

In other embodiments, any adjusted OD₆₀₀ measurement greater than blank OD₆₀₀ measurement can be determined as indicative of bacterial growth and applied as a threshold value by correlation to a standard reference method or combination of reference methods.

Minimal inhibitory concentrations for each effective antibiotic are then calculated based on the sensitivity or resistance of the culture at the multiple antibiotic concentrations.

Results of the antibiotic resistance assay disclosed herein are transmitted to the appropriate medical professional who then has the option of prescribing an antibiotic, or antibiotics, shown to be active against the patient's infection, changing the antibiotic to a more effective antibiotic, or ordering additional testing.

### Example 6. Antibiotic Resistance (ABR) Assay Utilizing Agar-Containing Medium

Urine samples suitable for processing with this assay are collected, transported, and stored using BD Vacutainer (gray top) tubes or other suitable leak-proof sterile container. Urine samples may be held at room temperature for 48 hours before test results are compromised.

Antibiotics not received in ready-made solutions were dissolved in appropriate solvent and according to their individual solubility (e.g., at 10× the concentration desired in the assay as antibiotic stocks). Antibiotic stocks are stored at 2-8° C. and protected from direct sunlight. Prepared antibiotic stock solutions were aliquoted into one or more 96-deep well plates (Thermo Fisher Scientific) to form an Antibiotic Source Plate, e.g., shown in FIG. 14 and identified by antibiotic name and concentration (μg/mL; 10× final concentration). Antibiotics may include nitrofurantoin, ciprofloxacin, meropenem, ceftriaxone, trimethoprim, sulfamethoxazole, trimethoprim/sulfamethoxazole, piperacillin, tazobactam, piperacillin/tazobactam, levofloxacin, cefoxitin, tetracycline, ampicillin/sulbactam, ampicillin, sulbactam, amoxicillin, amoxicillin/clavulanic acid, cefaclor, cefazolin, cefepime, ceftazidime, fosfomycin, gentamicin, and/or vancomycin, either singly or in combination. In some embodiments, one or more wells are designated AB-blend which contained a combination of antibiotics to ensure there was no bacterial growth.

Mueller-Hinton agar medium (e.g., 100 microliters) was aliquoted into each appropriate well position of a 96-well microplate (VIS 96/F-PS, Eppendorf). The medium was allowed to solidify at room temperature for at least 10 min.

The antibiotics (10 μL) at various concentrations were then aliquoted into desired wells from the Antibiotic Source Plate. After the antibiotics were introduced to the agar medium, the ABR microplates were allowed to sit for at least 1 hr before use. If long-term storage is required, ABR microplates containing antibiotic-infuse agar are stored at 2-8° C. in the dark.

At the time of testing, urine samples were diluted 1:20 in sterile saline and vortexed. Each patient sample utilized a single ABR microplate. Five microliters of diluted patient sample were added to each well of the room temperature microplate, the plate was sealed and incubated for 16-18 hr at 37° C.

After incubation, the plate was removed from the incubator and carefully uncovered. Two-hundred microliters of deionized water were added to each well to suspend cells present above the agar and the plates incubated at room temperature for 30 min. After 30 min, 100 μl from each well was removed to a new plate and the OD₆₀₀ was determined in a spectrophotometer. Five separate reads were taken of each plate and a mean OD₆₀₀ measurement calculated. The present invention is not limited to the use of OD to determine antibiotic resistance.

Controls include: No-antibiotic control, Negative control plate, and AB-Blend. No antibiotic control: Any well containing medium that is not infused with antibiotics to ensure viability of bacterial cells present in patient urine samples and included in each plate. If the no-antibiotic control for any given patient does not yield growth, a secondary test is performed using the same patient sample without dilution. Negative control plate: Microplate containing antibiotic-infused agar medium without addition of patient sample or cultured bacterial organisms to ensure non-contamination of reagents. AB-Blend: One or more wells containing a combination of antibiotics to ensure there is no bacterial growth.

Table 7 shows the antibiotics, well positions, mean OD (raw data), blanked OD, and an assessment of resistance (R) or susceptibility (S) to the antibiotic. Each well position corresponds to a particular antibiotic at a certain concentration. The wells in Table 7 are arranged by sorting like antibiotics together. The blanked column refers to the raw data being blanked by using the measurement obtained from the AB-Blend well, as depicted in Table 7. Raw data collected is shown as “mean” OD. To determine whether bacterial organisms present in the patient samples were resistant or sensitive to a particular antibiotic at a certain concentration, blanked OD readings were compared to a threshold OD₆₀₀ of 0.025. Any OD measurement greater than or equal to this threshold was designated Resistant (R) meaning bacterial organisms present in patient sample were resistant to that particular antibiotic at that certain concentration. Any OD measurement less than this threshold was designated Sensitive (S) meaning bacterial organisms present in patient sample were sensitive to that particular antibiotic at that certain concentration.

TABLE 7

| Antibiotic | Well | Mean | Blanked | Result |
|---|---|---|---|---|
| No-antibiotic | A7 | 0.6960 | 0.6576 | R |
| Nitro-32 | B7 | 0.0387 | 0.0003 | S |
| Nitro-64 | C7 | 0.0386 | 0.0002 | S |
| Nitro-128 | D7 | 0.0392 | 0.0008 | S |
| Cipro-1 | E7 | 0.0408 | 0.0024 | S |
| Cipro-2 | F7 | 0.0414 | 0.0030 | S |
| Cipro-4 | G7 | 0.0387 | 0.0003 | S |
| Mero-1 | H7 | 0.0392 | 0.0008 | S |
| Mero-2 | A8 | 0.0388 | 0.0004 | S |
| Mero-4 | B8 | 0.0390 | 0.0006 | S |
| Mero-8 | C8 | 0.0385 | 0.0001 | S |
| Amp/Sulb-8, 4 | D8 | 0.5372 | 0.4988 | R |
| Amp/Sulb-16, 8 | E8 | 0.5029 | 0.4646 | R |
| Amp/Sulb-32, 16 | F8 | 0.2925 | 0.2541 | R |
| Levo-1 | G8 | 0.0408 | 0.0024 | S |
| Levo-2 | H8 | 0.0401 | 0.0017 | S |
| Levo-4 | A9 | 0.0744 | 0.0360 | R |
| Levo-8 | B9 | 0.0412 | 0.0028 | S |
| Ceftria-1 | C9 | 0.0445 | 0.0061 | S |
| Ceftria-2 | D9 | 0.0432 | 0.0048 | S |
| Ceftria-4 | E9 | 0.0405 | 0.0021 | S |
| Ceftria-8 | F9 | 0.0392 | 0.0008 | S |
| Ceftria-16 | G9 | 0.0391 | 0.0007 | S |
| Ceftria-32 | H9 | 0.0386 | 0.0002 | S |
| Ceftria-64 | A10 | 0.0385 | 0.0001 | S |
| Vanco-2 | B10 | 0.4250 | 0.3866 | R |
| Vanco-4 | C10 | 0.4296 | 0.3912 | R |
| Vanco-8 | D10 | 0.4377 | 0.3993 | R |
| Vanco-16 | E10 | 0.4918 | 0.4534 | R |
| Vanco-32 | F10 | 0.4378 | 0.3994 | R |
| Pip/Tazo-16, 4 | G10 | 0.0495 | 0.0111 | S |
| Pip/Tazo-32, 4 | H10 | 0.0474 | 0.0090 | S |
| Pip/Tazo-64, 4 | A11 | 0.0477 | 0.0093 | S |
| Pip/Tazo-128, 4 | B11 | 0.0449 | 0.0065 | S |
| Cefox-4 | C11 | 0.0401 | 0.0017 | S |
| Cefox-8 | D11 | 0.0392 | 0.0008 | S |
| Cefox-16 | E11 | 0.0389 | 0.0005 | S |
| Cefox-32 | F11 | 0.0389 | 0.0005 | S |
| Tetra-4 | G11 | 0.4304 | 0.3920 | R |
| Tetra-8 | H11 | 0.4396 | 0.4012 | R |
| Tetra-16 | A12 | 0.4550 | 0.4166 | R |
| Amp-8 | B12 | 0.4880 | 0.4496 | R |
| Amp-16 | C12 | 0.5222 | 0.4838 | R |
| Amp-32 | D12 | 0.4824 | 0.4440 | R |
| TMP/SMX-2, 38 | E12 | 0.5087 | 0.4703 | R |
| TMP/SMX-4, 76 | F12 | 0.5307 | 0.4923 | R |
| AB-Blend | G12 | 0.0384 | 0 | S |
| empty | H12 | 0.7874 | 0.7490 | R |

In this example, the sample contains bacteria sensitive to nitrofurantoin, ciprofloxacin, meropenem, ceftriaxone, piperacillin/tazobactam, and cefoxitin. The results for levo are equivocal.

The MIC for each drug can then be provided. The minimum inhibitory concentration (MIC) is the minimum test antibiotic concentration to which the sample is sensitive. An exemplary MIC determination for meropenem based on the results above is depicted in Table 8.

TABLE 8

| 1 | 2 | 3 | 4 | 5 | 7 | |
|---|---|---|---|---|---|---|
| ero [1] | ero [2] | ero [4] | ero [8] | IC | Interpretation | |
| 6 | 7 | 8 | 9 | 10 = 1 | 11 | S |
| 12 | 13 | 14 | 15 | 16 = 2 | 17 | I |
| 18 | 19 | 20 | 21 | 22 = 4 | 23 | I |
| 24 | 25 | 26 | 27 | 28 = 8 | 29 | I |
| 30 | 31 | 32 | 33 | 34 = 8 | 35 | R |

Accuracy was assessed by comparing the antibiotic resistance results of the test method compared to those obtained for mixed and isolated cultures evaluated by the antibiotic-agar method. A total of 19 bacterial pools (pools consist of 2-4 organisms), 17 isolated organisms, and 9 routinely processed urine samples were tested for resistance to 12 antibiotics. Accuracy was assessed in regards to Specificity (True Negatives), Sensitivity (True Positives), and Overall Accuracy (All Samples). The assay showed good accuracy in all three categories: overall accuracy=96%; % accuracy for specificity=95%; % accuracy for sensitivity=96%.

Inter-assay precision was evaluated by testing three samples from the “Accuracy” sample set over three days. Intra-assay precision was evaluated by testing each of these samples in triplicate in one batch. Precision for each sample was assessed by determining the consensus result of all 5 replicates and then counting the number of replicates that match the consensus. This number was then divided by the sum of all measurements (sum of measurements for all drugs) to determine the % precision. The overall precision was calculated by dividing the sum of all correct matches by the total number of measurements from all samples. The assay demonstrated very good precision. The total matched of all precision samples was 643 out of 690 measured, a percentage of 93%.

Analytic sensitivity, or the limit of detection (LOD), was assessed by determining the lowest bacterial concentration that yielded accurate results. In certain cases, bacterial concentrations lower than 10,000 cells/mL are not considered positive for UTI and therefore the lowest concentration tested was 10,000 cells/mL. Consistent results (>98%) correlation to the consensus results were obtained at the lowest bacterial concentrations tested. The LOD of this assay was 10,000 cells/mL. Note, the present invention is not limited to a concentration of 10,000 cells/mL.

The analytic specificity of this assay was assessed by testing samples at bacterial concentrations of 100,000,000 cells/mL. Such concentrations are not typically observed in routine UTI patient samples but were achieved in saturated overnight bacterial cultures. Assessment of analytic measurement range (AMR) was then performed by testing three samples from the “Accuracy” sample set each diluted as follows: 100,000,000 cells/mL, 1,000,000 cells/mL, 100,000 cells/mL and 10,000 cells/mL. Consistent results (>94%) correlation to the consensus results were obtained at all bacterial concentrations tested. The assay is specific at bacterial concentration up to 100,000,000 cells/mL. The present invention is not limited to a concentration of 10,000 cells/mL.

### Example 7. Antibiotic Resistance (ABR) Assay Utilizing Liquid Growth Medium

Urine samples suitable for processing with this assay are collected, transported, and stored using BD Vacutainer tubes or other suitable leak-proof sterile containers. Urine samples may be held at room temperature for 48 hours before test results are compromised.

Antibiotics not received in ready-made solutions were dissolved in appropriate solvents and according to their individual solubility to 50× the concentration desired in the assay and stored as antibiotic stocks. Antibiotic stocks are stored at 2-8° C. and protected from direct sunlight. Prepared antibiotic stock solutions were aliquoted into a 96-deep well plate (ThermoFisher Scientific) to form a 50× Antibiotic Source Plate and then diluted 1:5 to form a 10× Antibiotic Source Plate, as shown in FIG. 15 where each well is identified by antibiotic name and concentration (μg/mL; 10× final concentration). Antibiotics included in this assay were amoxicillin, clavulanate, ampicillin, sulbactam, cefaclor, cefazolin, cefepime, cefoxitin, ceftazidime, ceftriaxone, ciprofloxacin, fosfomycin, gentamicin, levofloxacin, meropenem, nitrofurantoin, piperacillin, tazobactam, tetracycline, trimethoprim, sulfamethoxazole, and vancomycin, either singly or in combination. One well was assigned sodium azide to ensure no bacterial growth would be observed in that well.

Twenty microliters of each antibiotic solution were aliquoted into the pre-determined wells of a 96-well microplate (VIS 96/F-PS, Eppendorf) from the 10× Antibiotic Source Plate to create ABR testing plates for inoculation. These ABR testing plates were allowed to sit for up to 24 hours before use at 2-8° C. in the dark.

At the time of testing, urine samples were centrifuged to concentrate any bacterial cells and then mixed with liquid Mueller-Hinton medium and incubated for 6-16 hours at 37° C. After this initial incubation, the sample is diluted to 0.5-0.6 McF in saline and then 500 μl of that suspension was added to 29.5 μl of Mueller-Hinton medium. One-hundred and eighty microliters of the diluted sample is then aliquoted to each well of the ABR microplate already containing 10× antibiotic solution, bringing all of the antibiotics to the desired final concentration. The plate is then sealed and incubated for 12-16 hours at 37° C.

After incubation, the plate was removed from the incubator and carefully uncovered and the OD₆₀₀ was determined for each appropriate well by spectrophotometer. Five separate measurements were taken of each well on a plate and the mean OD₆₀₀ measurement calculated for each well. Controls include (1) no-antibiotic control: Any well containing medium that is not infused with antibiotics to ensure viability of bacterial cells present in patient urine samples and included in each plate; If the no-antibiotic control for any given patient does not yield growth, the sample is repeated on the assay and reported as quantity not sufficient if repeat testing still does not yield satisfactory results; (2) negative control plate: Microplate containing antibiotic-infused agar medium without addition of patient sample or cultured bacterial organisms to ensure non-contamination of reagents; and (3) Na Azide: One or more wells containing a dilute concentration of sodium azide to ensure no bacterial growth will occur.

Table 9 shows the antibiotics, well positions, mean OD (raw data), blanked OD, and an assessment of resistance (R) or susceptibility (S) to the antibiotic. Each well position corresponds to a particular antibiotic at a certain concentration. The wells are arranged by sorting like antibiotics together. The blanked column refers to the raw data being blanked by using the measurement obtained from the Na Azide well. Raw data collected is shown as “mean” OD. To determine whether bacterial organisms present in the patient samples were resistant or sensitive to a particular antibiotic at a certain concentration, blanked OD readings were compared to a threshold OD₆₀₀ of 0.065. An OD measurement greater than or equal to this threshold was designated Resistant (R) meaning bacterial organisms present in patient sample were resistant to that particular antibiotic at that certain concentration. Any OD measurement less than this threshold was designated Sensitive (S) meaning bacterial organisms present in patient sample were sensitive to that particular antibiotic at that certain concentration.

TABLE 9

| Antibiotic | Well | Mean | Blanked | Result |
|---|---|---|---|---|
| Na Azide | E8 | 0.0457 | 0 | S |
| No-Antibiotic | A1 | 0.2217 | 0.1760 | R |
| No-Antibiotic | F8 | 0.2604 | 0.2147 | R |
| No-Antibiotic | G8 | 0.2354 | 0.1897 | R |
| No-Antibiotic | H8 | 0.2446 | 0.1989 | R |
| Amox/Clav-8, 4 | G7 | 0.0418 | −0.0039 | S |
| Amox/Clav-32, 16 | H7 | 0.0437 | −0.0020 | S |
| Amp-8 | B5 | 0.0417 | −0.0040 | S |
| Amp-16 | C5 | 0.0419 | −0.0038 | S |
| Amp-32 | D5 | 0.0418 | −0.0039 | S |
| Amp/Sulb-8, 4 | B2 | 0.0413 | −0.0044 | S |
| Amp/Sulb-32, 16 | C2 | 0.0414 | −0.0043 | S |
| Cefaclor-8 | C8 | 0.2332 | 0.1875 | R |
| Cefaclor-32 | D8 | 0.0600 | 0.0143 | S |
| Cefazolin-2 | G5 | 0.2339 | 0.1882 | R |
| Cefazolin-8 | H5 | 0.2090 | 0.1633 | R |
| Cefazolin-16 | A6 | 0.1468 | 0.1011 | R |
| Cefazolin-32 | B6 | 0.0570 | 0.0113 | S |
| Cefepime-1 | C6 | 0.2416 | 0.1959 | R |
| Cefepime-2 | D6 | 0.2427 | 0.1970 | R |
| Cefepime-4 | E6 | 0.2417 | 0.1960 | R |
| Cefepime-8 | F6 | 0.2404 | 0.1947 | R |
| Cefepime-16 | G6 | 0.2323 | 0.1866 | R |
| Cefepime-32 | H6 | 0.2281 | 0.1824 | R |
| Cefoxitin-4 | C4 | 0.2202 | 0.1745 | R |
| Cefoxitin-8 | D4 | 0.2288 | 0.1831 | R |
| Cefoxitin-32 | E4 | 0.2348 | 0.1891 | R |
| Ceftazidime-4 | A7 | 0.2457 | 0.2000 | R |
| Ceftazidime-8 | B7 | 0.2607 | 0.2150 | R |
| Ceftazidime-16 | C7 | 0.2473 | 0.2016 | R |
| Ceftazidime-32 | D7 | 0.2437 | 0.1980 | R |
| Ceftriaxone-1 | H2 | 0.2485 | 0.2028 | R |
| Ceftriaxone-4 | A3 | 0.2357 | 0.1900 | R |
| Ceftriaxone-8 | B3 | 0.2449 | 0.1992 | R |
| Ceftriaxone-64 | C3 | 0.2356 | 0.1899 | R |
| Cipro-1 | D1 | 0.0441 | −0.0016 | S |
| Cipro-4 | E1 | 0.0423 | −0.0034 | S |
| No-Antibiotic | A8 | 0.0427 | −0.0030 | S |
| No-Antibiotic | B8 | 0.0419 | −0.0038 | S |
| Gentamicin-4 | E7 | 0.0615 | 0.0158 | S |
| Gentamicin-16 | F7 | 0.0426 | −0.0031 | S |
| Levo-1 | D2 | 0.1180 | 0.0723 | R |
| Levo-2 | E2 | 0.0436 | −0.0021 | S |
| Levo-4 | F2 | 0.0431 | −0.0026 | S |
| Levo-8 | G2 | 0.0443 | −0.0014 | S |
| Mero-1 | F1 | 0.2198 | 0.1741 | R |
| Mero-2 | G1 | 0.1928 | 0.1471 | R |
| Mero-4 | H1 | 0.1556 | 0.1099 | R |
| Mero-8 | A2 | 0.0496 | 0.0039 | S |
| Nitro-32 | B1 | 0.0552 | 0.0095 | S |
| Nitro-128 | C1 | 0.0539 | 0.0082 | S |
| Pip/Tazo-16, 4 | A4 | 0.0421 | −0.0036 | S |
| Pip/Tazo-128, 4 | B4 | 0.0419 | −0.0038 | S |
| Tetra-2 | F4 | 0.2084 | 0.1627 | R |
| Tetra-4 | G4 | 0.2224 | 0.1767 | R |
| Tetra-8 | H4 | 0.1596 | 0.1139 | R |
| Tetra-16 | A5 | 0.0539 | 0.0082 | S |
| TMP/SMX-2, 38 | E5 | 0.1209 | 0.0752 | R |
| TMP/SMX-4, 76 | F5 | 0.1016 | 0.0559 | S |
| Vanco-1 | D3 | 0.0504 | 0.0047 | S |
| Vanco-2 | E3 | 0.0435 | −0.0022 | S |
| Vanco-4 | F3 | 0.0425 | −0.0032 | S |
| Vanco-16 | G3 | 0.0431 | −0.0026 | S |
| Vanco-32 | H3 | 0.0426 | −0.0031 | S |

In this example, the sample contains bacteria sensitive to amoxicillin/clavulanate, ampicillin, ampicillin/sulbactam, ciprofloxacin, gentamicin, levofloxacin, nitrofurantoin, piperacillin/tazobactam, and vancomycin.

The MIC for each drug can then be provided. The minimum inhibitory concentration (MIC) is the minimum test antibiotic concentration to which the sample is sensitive. An exemplary MIC determination for meropenem based on the results above is depicted in Table 10.

TABLE 10

| 40 | 41 | 42 | 43 | 44 | 45 | |
|---|---|---|---|---|---|---|
| ero [1] | ero [2] | ero [4] | ero [8] | IC | Interpretation | |
| 46 | 47 | 48 | 49 | 50 = 1 | 51 | S |
| 52 | 53 | 54 | 55 | 56 = 2 | 57 | I |
| 58 | 59 | 60 | 61 | 62 = 4 | 63 | I |
| 64 | 65 | 66 | 67 | 68 = 8 | 69 | I |
| 70 | 71 | 72 | 73 | 74 = 8 | 75 | R |

Accuracy was assessed by comparing the antibiotic resistance results of the test method to a consensus of results obtained by standard reference methods. A total of 15 isolated organisms, and 20 routinely processed patient urine samples were tested for resistance to 18 antibiotics, each tested at multiple concentrations for a total of 57 antibiotic concentrations. Accuracy was assessed regarding Specificity (True Negatives), Sensitivity (True Positives), and overall Accuracy (all samples). The assay showed good accuracy in all three categories: overall accuracy=96%; specificity=95%; sensitivity=97%.

Inter-Assay precision was evaluated by testing five samples over three different days. Intra-Assay precision was evaluated by testing the same five samples in triplicate in a single day. Percent concordance was calculated to measure the precision of results obtained by this assay. The assay demonstrated very good precision. In the Intra-assay, the number of matches was 841 out of 855 measurements (98% concordance); for the Inter-assay, the number of matches was 1388 out of 1425 measured (97% concordance).

Analytic sensitivity was evaluated by creating a dilution series of E. coli and E. faecalis with the lowest bacterial concentration at less than 100 cells/mL for each organism. Each dilution level for each isolate was tested to show reproducibility of results down to the lowest concentration. 98% correlation was observed across all dilution levels for both isolates, indicating the limit of detection (LOD) of this assay is less than 100 cells/ml.

Analytic specificity was evaluated in the context of inhibitory effect of overloading the assay with too many bacterial cells. Lower accuracy (due to false-resistant results) was observed for samples inoculated at high bacterial concentration. This indicates that all samples must be diluted to the specified cell density post pre-culture and before ABR inoculation.

This assay utilizes a pre-culture step prior to introducing samples to antibiotics. The duration of this pre-culture incubation was tested at 6 and 16 hours for 2 isolates (E. coli and E. faecalis). Good accuracy for each isolate was observed after both 6 and 16 hour pre-culture incubations, indicating a pre-culture window of 6 to 16 hours for this assay. The number of matches was 81 out of 83 measurements (98% accuracy).

Once samples are introduced to antibiotics, they are incubated for 12 to 16 hours. This incubation length was determined by obtaining OD measurements for Precision samples after 12 and 16 hours of incubation. Good percent concordance was observed for all samples across within a 12 to 16 hour incubation window. The number of matches was 2758 out of 2850 measurements (97% accuracy).

To confirm turbidity (high OD measurements) are due to bacterial growth, DNA was extracted from wells corresponding to Sensitive and Resistant results and tested for pathogen identification by PCR. Identification results confirm Resistant (turbid) wells contained significantly higher bacterial concentration than Sensitive (clear) wells (see Table 11).

TABLE 11
Overall (Cells/mL)

| | |
|---|---|
| Resistant | 5,170,897,798 |
| Sensitive | 1,341,116 |
| Fold-Diff | 3,856 |

### Example 8: Concordance Between the Presence of Antibiotic Resistance Genes by Multiplex PCR and Susceptibility Testing in Symptomatic Patients with Urinary Tract Infection

There are two mechanisms of antibiotic resistance: innate resistance and acquired resistance. Innate antibiotic resistance is usually chromosome-encoded, such as the non-specific efflux pumps, antibiotic inactivating enzymes, or permeability barriers. Horizontally transferred resistant genes provide acquired resistance and include plasmid encoded ABR genes for specific efflux pumps and enzymes that can modify targeted antibiotics.

While an increasing number of ABR genes are known, detection of an ABR gene does not guarantee the activity of that gene. Since the regulation of many of these ABR genes has not been fully understood, further research is needed to understand how frequently the presence of an ABR gene is correlated with its activity. The present invention analyzes the concordant and discordant rates between the presence or absence of ABR genes and antibiotic susceptibility testing in urine samples collected from UTI-symptomatic patients.

This concordance study was based on a subset of a prospective UTI study cohort of 2,512 consecutive patients enrolled between Jul. 26, 2018, and Feb. 27, 2019. Briefly, patients presenting with UTI symptoms were evaluated by 75 physicians from 37 urology clinics across the United States. The study included patients 60 years or older presenting at the urology office with a suspicion of acute cystitis, complicated UTI, persistent UTI, recurrent UTIs, prostatitis, or pyelonephritis. Additionally, the study also included patients at any age, presenting with a history of interstitial cystitis. Patients were excluded if they lacked UTI symptoms, took antibiotics use for any reason other than a UTI at the time of enrollment, patients with urinary diversion, and the use of chronic 10 days) indwelling catheter or self-catheterization at the time of consultation. Patients with urine samples without proper documentation of the specimen collection method, did not meet the collection criteria for testing, or did not contain at least 3 mL of volume were excluded from the study. All patients provided written informed consent with forms approved by the Western IRB (20181661).

From each patient 3 ml of urine was used for bacterial detection by M-PCR (methods below). Among the 2,512 patients, M-PCR detected bacteria in a total of 1,579 patients. Three hundred and seventy-two of the patient samples contained exclusively fastidious bacteria that were deemed unculturable by lab standards; as a result, susceptibility testing could not be performed. Pooled antibiotic susceptibility testing (P-AST) results were not available for 52 patients because the bacteria failed to thrive during testing. Therefore, the concordance analysis data were based on a subset (N=1,155) of the total study cohort. This subset only includes patients with positive bacterial identifications by M-PCR, antimicrobial susceptibility results from P-AST, and results from the ABR detection by M-PCR.

DNA was extracted from urine samples with the KingFisher/MagMAX Automated DNA Extraction instrument and the MagMAX DNA Multi-Sample Ultra Kit (Thermo Fisher, Carlsbad, CA). Briefly, 400 μL of urine was transferred to 96-deep-well plates, sealed, and centrifuged to concentrate the samples; the supernatant was removed. Enzyme Lysis Mix (220 μL/well) was added and incubated for 20 min at 65° C. Proteinase K Mix was added (50 μL/well) and incubated for 30 min at 65° C. Lysis buffer (125 μL/well) and DNA Binding Bead Mix (40 μL/well) were added, and the samples were shaken for a minimum of 5 min. The 96-well plate was loaded into the KingFisher/MagMAX Automated DNA Extraction instrument, according to standard operating procedures.

The presence of bacteria was determined using the Pathnostics Guidance® UTI Test, as described previously. Briefly, the DNA extracted from patient samples were mixed with a universal PCR master mix and amplified with TaqMan technology on a Life Technologies 12K Flex Open Array System. DNA samples were spotted in duplicate on 112-format OpenArray chips. Positive controls were included in the form of plasmids containing bacterial target DNA. Candida tropicalis was used as an inhibition control. A data analysis tool developed by Pathnostics was used to sort data, assess the quality of data, summarize control sample data, identify positive assays, calculate concentrations, and generate results.

The following bacteria and bacterial groups were detected using the Pathnostics Guidance® UTI Test. Not all organisms detected by this test were readily cultivatable using standard culture protocols. Therefore, the 16 bacteria listed in bold were included in P-AST and the concordance analyses: Acinetobacter baumannii, Actinotignum schaalii, Aerococcus urinae, Alloscardovia omnicolens, Citrobacter freundii, Citrobacter koseri, Corynebacterium riegelii, Klebsiella aerogenes, Enterococcus faecalis, Escherichia coli, Klebsiella oxytoca, Klebsiella pneumoniae, Morganella morganii, Mycoplasma genitalium, Mycoplasma hominis, Pantoea agglomerans, Proteus mirabilis, Providencia stuartii, Pseudomonas aeruginosa, Serratia marcescens, Staphylococcus aureus, Streptococcus agalactiae, Ureaplasma urealyticum, Coagulase negative staphylococci group (CoNS), and Viridans group streptococci (VGS). Patient samples with any of the 16 bacteria identified were analyzed in P-AST and in the concordance analyses.

The quantities of each of the bacterial species were determined using the standard curve method, as described previously. Briefly, standard curves of each of the bacteria were generated from testing replicates of dilution series of DNA/culture at known concentrations; constants necessary for the quantitation of each of the bacterial species in unknown samples were established from the standard curves. PCR cycle values of a target bacterium from a patient sample were compared to the standard curve, and the concentration of the target bacterial species (cells/mL) present in the samples was extrapolated and determined. A bacterium with a quantity of 10,000 cells/mL was defined as “positive” or “detected.” and bacteria with quantity <10,000 cells/mL were defined as “negative”. The present invention is not limited to these thresholds.

A total of 33 ABR genes linking to the resistances of 6 classes of antibiotics were tested in using the Pathnostics Guidance® UTI Test including carbapenem resistance genes (VIM, KPC, IMP-1 group, IMP-7, OXA-72, OXA-23, OXA-40, OXA-48, OXA-58), ampicillin resistance genes (DHA, MOX/CMY, BIL/LAT/CMY, AmpC, FOX, ACC), fluoroquinolone resistance genes (QnrA, QnrB), vancomycin resistance genes (vanA1, vanA2, vanB), extended-spectrum beta-lactamases (ESBL) resistance genes (CTX-M group 1, CTX-M group 2, CTX-M group 8/25, CTX-M group 9, PER-1, PER-2, NDM-1, OXA-1, GES, SHV, TEM, and VEB), and one methicillin resistance gene (mecA). Each spot of a 112-format OpenArray chip was coated with a probe for a single gene with the exception of three groups which were combined to coat a single spot (spot 1: DHA/MOX/CMY; spot 2: BIL/LAT/CMY; and spot there: AmpC/FOX/ACC). Two additional ABR genes, ErmA and ErmB, associated with resistance to macrolide class of antibiotics were also included on the OpenArray chip as part of the ABR gene testing. However, results from these two genes were not included in the concordance analysis as no macrolide antibiotics were part of the P-AST testing.

A bacterium was determined to be positive for an ABR gene if the cycle number (Ct) of that ABR gene was above a particular threshold. That threshold was determined by comparing a series of negative samples, extraction control samples, and specificity samples (genomic DNA of non-target organism/gene). The lowest Ct from these assays was determined. Second, a plasmid dilution series was tested (ThermoFisher provided plasmids for each target ABR gene). The lowest plasmid concentration in which 50% or more of the replicates were detected with a Ct value below the cycle number determined in the first step was established as the lower limit of detection (LLOD) for an assay. The threshold Ct for any particular target ABR gene assay was then set at the cycle equivalent of the determined LLOD for that gene. ABR genes with Ct value no higher than the Ct threshold of the gene was defined as “positive” or “detected”, and ABR genes with Ct value higher than the threshold was defined as “negative” or “not detected.” For mecA gene, “positive” or “detected” status for mecA was only limited to patients with S. aureus detection.

The present invention is not limited to the thresholds disclosed herein.

A total of 18 antibiotics representing six antibiotic classes (aminopenicillins, beta-lactamase inhibitor combinations, cephalosporins, fluoroquinolones, carbapenems, and glycopeptides) were evaluated in the P-AST assay. Four antibiotics were not associated with any of the ABR genes on the Pathnostics Guidance® UTI Test detection panel. Therefore, 14 of the 18 antibiotics were evaluated in the concordance analysis. The antibiotics were purchased from Sigma Aldrich: amoxicillin/clavulanate (Cat. #A8523-1G/33454-100MG), ampicillin (Cat. #A5354-10ML), ampicillin/sulbactam (Cat. #A5354-10ML/1623670-250MG), cefaclor (Cat. #PHR1283-1G), cefazolin (Cat. #C5020-1G), cefepime (Cat. #PHR1763-1G), cefoxitin (Cat. #C4786-1G), ceftazidime (Cat. #A6987-5G), ceftriaxone (Cat. #C5793-1G), ciprofloxacin (Cat. #17850-5G-F), levofloxacin (Cat. #28266-10G-F), meropenem (Cat. #M2574-10MG), piperacillin/tazobactam (Cat. #P8396-1G/1643383-200MG), and vancomycin (Cat. #SBR00001-10ML).

P-AST was performed as described previously. Briefly, 1 mL of patient urine sample was aliquot into a 1.7 mL microcentrifuge tube. After centrifugation, the supernatant was aspirated and discarded, leaving approximately 500 μL of the patient sample in the microcentrifuge tube. One mL of Mueller Hinton Growth Media was then aliquot into the sample in the microcentrifuge tube and the tubes were incubated at 35° C. in a non-CO₂ incubator for 6 hours. Samples that reached a minimum threshold of 10,000 cells/mL were diluted by aliquoting 0.5 mL of sample into a 50 mL conical tube containing Mueller Hinton Growth Media. 96-well plates pre-loaded with dilution series of different antibiotics were inoculated with samples and incubated along with control plates for 12-16 hours at 35° C. aerobically/anaerobically in a single layer. A DensiCHEK plate Reader™ (BioMerieux, Marcy-I'Étoile, France) was used to measure optical densities every X hours/min. Growth curves of cultures grown with antibiotics were compared to standard growth to determine the resistance to a given antibiotic.

The demographic and clinical information for the patients, along with frequencies detection of bacterial species, presence or absence of ABR gene, and antibiotic resistance rates were summarized for the entire study cohort. For each antibiotic, ABR genes' status was coded as “present” if any one or more of the associated ABR genes were detected. The present invention is not limited to the thresholds disclosed herein.

The concordant rates between the presence of ABR genes and the susceptibility test results were generated for each antibiotic, each antibiotics class, monomicrobial infections, polymicrobial infections, and the overall group. The concordant rates for each antibiotic between monomicrobial and polymicrobial samples were compared using the chi-square test. The analyses were performed using SAS version 9.4.

Of the 1,155 patients included in the concordance analyses, 784 (67.9%) were females, and 371 (32.1%) were males. The median age was 74.3 years, with 99.3% over the age of 60. All patients presented with UTI symptoms, including dysuria, cloudy or strong-smelling urine, pain or pelvic discomfort, fever, and/or lower urinary tract symptoms (LUTS). Urinalysis was positive for blood, leukocytes and/or nitrites in 89.0% of the study population. Fourteen-point three percent of the patients were on an antibiotic within the three weeks before enrollment into the study.

TABLE 12

| | Antibiotic | Detection of associated |
|---|---|---|
| Antibiotics | Resistance I | ABR Genes (%) |
| Amoxicillin/Clavulanate | 29.1% | 36.8% |
| Ampicillin | 48.9% | 30.1% |
| Ampicillin/Sulbactam | 31.8% | 36.6% |
| Cefaclor | 49.3% | 35.7% |
| Cefazolin | 38.2% | 35.8% |
| Cefepime | 40.9% | 35.7% |
| Cefoxitin | 44.9% | 36% |
| Ceftazidime | 46.3% | 35.7% |
| Ceftriaxone | 50.9% | 36.5% |
| Ciprofloxacin | 35.4% | 0.3% |
| Levofloxacin | 29.7% | 0.3% |
| Meropenem | 24.1% | 0.3% |
| Piperacillin/Tazobactam | 6.2% | 35.7% |
| Vancomycin | 44.1% | 0.8% |

M-PCR detected a polymicrobial infection, defined as two or more bacteria, in 23.3% (269) of patients, and 886 (76.7%) patients contained one microorganism. Overall, the most detected bacteria in patients' urine samples were E. coli (564/1,155, 48.8%), CoNS group (282/1,155, 24.4%), and E. faecalis (242/1,155, 21.0%). Data not shown: the detection frequency of each of the bacteria detected.

The resistance rates for each antibiotic ranged from 6.2% (for piperacillin/tazobactam) to 50.9% (for ceftriaxone) (see Table 12). The detection rate for the ABR genes range from 0.3% (for meropenem, levofloxacin, and ciprofloxacin-associated ABR genes) to 36.8% (for amoxicillin/clavulanate-associated ABR genes)(see Table 12).

M-PCR detected 24 ABR genes 470 times in 36.2% (419/1155) of patient samples, the majority of whom [379/419 (90.5%)] had only one ABR gene detected. M-PCR detected two ABR genes in 29/419 (6.92%) of samples, and 3 ABR genes in 11/419 (2.63%) of samples. The most frequently identified ABR genes were TEM, SHV, and CTX-M group 1 gene, in 205 (17.7%), 100 (8.7%), and 47 (4.1%) patients, respectively. Nine ABR genes, including GES, IMP-1 group, PER-1, PER-2, IMP-7, OXA-72, OXA-58, NDM-1, and OXA-23, were not detected in any patient samples.

Three hundred seventy-two patients' samples exclusively contained eight fastidious bacteria of which, 26 (7.0%) were identified with presence of ABR genes. Twenty-four samples contained 1 ABR gene, 1 with two genes, and 1 with three genes, with a total of 15 genes identified. These patients were excluded from concordance analyses because P-AST results were not available. The ABR genes detected in the samples are associated with resistance to the following classes of antibiotics: aminopenicillins, beta-lactamase inhibitor/antibiotic combinations, glycopeptides, fluoquinolones, carbapenems, and cephalosporins. Data not shown: the prevalence of each of the ABR genes in the 372 patients.

An overall concordance rate of 60% was observed between ABR genes' presence by M-PCR and the antimicrobial susceptibility results by P-AST. There were two different types of concordance values: ABR gene was not present by M-PCR/antibiotic sensitive by P-AST (48.4%) and ABR gene present/antibiotic-resistant by P-AST (11.5%). The 40% non-concordant cases also included two circumstances: ABR gene not present/antibiotic-resistant (25%) and ABR gene present/antibiotic-sensitive (15%).

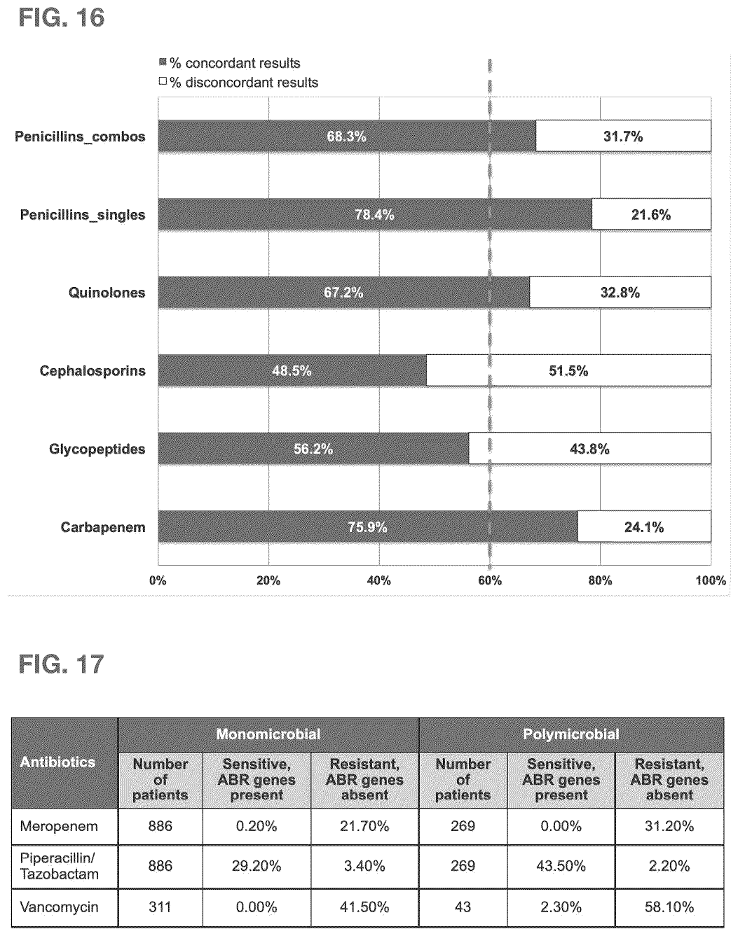

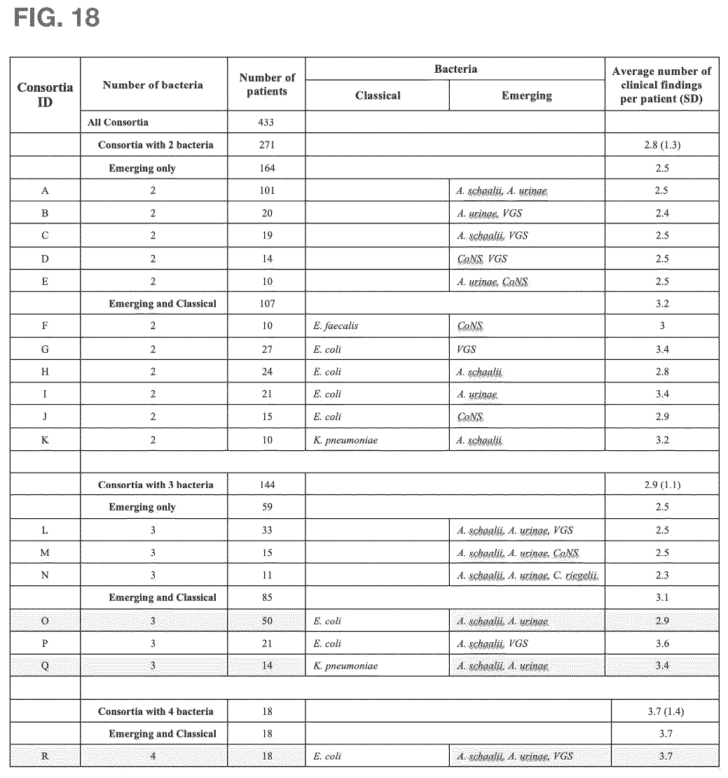

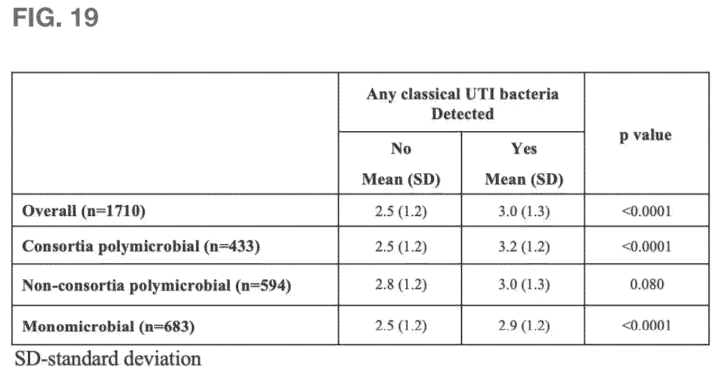

Some antibiotic categories showed higher concordance than the overall concordance rate. For example, aminopenicillins, beta-lactamase inhibitor/antibiotic combinations, fluoroquinolones, and carbapenems had concordance rates ≥67.2c/o. By contrast, cephalosporins only exhibited a concordance rate of 48.5% (see FIG. 16).

A total of 14 antibiotics were involved with the concordance analysis. The overall concordance rates for each of the antibiotics range from 44.7% (ceftriaxone) to 78.4% (ampicillin). Most antibiotics were associated with similar concordance rates for monomicrobial and polymicrobial infections. However, the concordance rates were significantly lower for polymicrobial for three antibiotics, including vancomycin, meropenem, and piperacillin/tazobactam, with absolute differences of 9.3% (p value=0.002), 13.1% (p value=<0.0001), and 19.0% (p value=0.019), respectively (see Table 13). The higher discordance rates for vancomycin and meropenem in the polymicrobial data set was due to an increased phenotypic resistant rate in the absence of ABR gene detection in the polymicrobial setting than in the monomicrobial setting. The higher discordance for piperacillin/tazobactam was due to an increased level of ABR gene detection in the presence of the sensitive phenotype.

TABLE 13

| | | | Concordance | Concordance | |
|---|---|---|---|---|---|
| | Number of | | rate in | rate in | |
| | associated | Concordance | Monomicrobial | Polymicrobial | p value |
| | ABR genes | rate in | specimens | specimens | (Monomicrobial vs. |
| Antibiotics | tested | All (N = 1155) | (N = 886) | (N = 269) | Polymicrobial) |
| Amoxicillin/ | 12 | 66.8% | 67.9% | 62.7% | 0.13 |
| Clavulanate | | | | | |
| Ampicillin | 12 | 78.4% | 79% | 75.7% | 0.34 |
| Ampicillin/ | 12 | 74% | 74.9% | 71% | 0.24 |
| Sulbactam | | | | | |
| Cefaclor | 12 | 48.3% | 48.9% | 46.3% | 0.45 |
| Cefazolin | 12 | 55.7% | 56.6% | 52.7% | 0.29 |
| Cefepime | 12 | 50.3% | 51.8% | 45.4% | 0.066 |
| Cefoxitin | 12 | 46.9% | 47.1% | 46.3% | 0.82 |
| Ceftazidime | 12 | 45.4% | 46% | 43.1% | 0.4 |
| Ceftriaxone | 12 | 44.7% | 43.4% | 49% | 0.12 |
| Ciprofloxacin | 2 | 64.4% | 65.3% | 61.3% | 0.23 |
| Levofloxacin | 2 | 70.1% | 71.3% | 66.2% | 0.11 |
| Meropenem | 6 | 75.9% | 78.1% | 68.8% | 0.002 |
| Piperacillin/ | 12 | 64.3% | 67.4% | 54.3% | <0.0001 |
| Tazobactam | | | | | |
| Vancomycin | 3 | 56.2% | 58.5% | 39.5% | 0.019 |

Antibiotic resistance, especially among uropathogens, is an increasingly important issue. Reliable and rapid microbial identification with resistance information is essential for the management of UTIs and antibiotic stewardship. M-PCR-based tests have been developed for clinical use for UTI in the detections of ABR genes. However, the detection of an ABR gene may not translate into phenotypic resistance, nor can their absence indicate susceptibility.

The study acquired over urine samples from 1,155 patients with UTI symptoms from 37 different urology clinics in the United States. The study used an M-PCR-based test to detect the presence of different bacteria and ABR genes, evaluated the antimicrobial susceptibility with a P-AST test, and measured the concordance between the two test results, e.g., ABR gene status (present or absent) and the antimicrobial susceptibility results from the P-AST test. The overall concordance rate between the two results was 60%, including 48% lacking the presence of ABR genes and producing sensitive P-AST results, and 12% showing the presence of ABR genes and producing resistant P-AST results. The 40% discordance included a 25% lack of ABR genes and resistant P-AST results and 15% of the presence of ABR genes and sensitive P-AST results.

Even though the 60% concordance further validated the ABR genes' roles in antibiotic resistance, the 40% discordance demonstrated the limitations of using the presence or absence of ABR genes alone to make clinical decisions. This current study used an M-PCR OpenArray chip to identify 35 ABR genes, yet there are thousands of ABR genes associated with hundreds of antibiotics in hundreds of bacteria. Incorporating all ABR genes into a single assay is impossible. Additionally, the number of ABR genes being discovered is increasing due to ongoing research. Therefore, the antibiotic resistance detected may, in part, result from a mechanism involving ABR genes not included in the testing panel.

On the other hand, in approximately 15% of situations, the presence of ABR genes did not confer phenotypic resistance. The discordance could be due to several reasons. The PCR assay detects the presence of the ABR gene at the DNA level. For an ABR gene to ultimately generate resistance, the bacterium must first transcribe the gene into messenger ribonucleic acid (mRNA). Next, the ribosomes translate the mRNA into a protein; then in some cases, the protein must be activated when signaled to do so. If mutations, for example, are in the gene promoter region, it may not produce a protein, thus, not yielding antibiotic resistance. Furthermore, mutational changes in the coding region of an ABR gene, such as a frame-shift mutation, may also fail to produce a downstream protein product of the ABR gene, preventing the bacteria from generating the antibiotic-resistant phenotype. Therefore, this study demonstrates that the presence or absence of the ABR gene alone is not entirely reliable in predicting bacterial antibiotic response and supports the clinical utility of antimicrobial susceptibility in aiding clinical treatment decision-making.

The concordance rate differed by antibiotic classes. For example, the concordance rates were as high as 78% for single-agent penicillin's, and as low 48.4% for cephalosporins. At the individual antibiotic level, there was a significant mismatch between ABR genes and P-AST results for five antibiotics, four of which included: meropenem, ciprofloxacin, levofloxacin, and vancomycin. The number of ABR genes targeted for detection for ciprofloxacin, levofloxacin, meropenem, and vancomycin were less than those targeted at the individual level for cephalosporins, beta-lactamase inhibitory combinations, and penicillin. Thus, it may be possible that other unknown ABR genes associated with resistance to ciprofloxacin, levofloxacin, meropenem, and vancomycin, are not included in the testing panel.

Piperacillin/tazobactam was the fifth antibiotic to display a high rate of discordance. In the case of piperacillin/tazobactam, there was a higher percentage of ABR genes identified than the rate of resistance from P-AST results. Resistance was detected in only 6.2% of cases, while 35.7% of cases showed ABR resistance genes. Cabolt et al. showed that resistance to piperacillin/tazobactam requires the overexpression of AmpC along with the assistance from two additional ABR genes, mexB and mexY, both of which were not targeted for in the study. Therefore, a significant portion of the 35.7% that was positive for ABR genes tested in the study may be negative for mexB or mexY, and not overexpress AmpC, and thus, failed to result in a resistant phenotype toward piperacillin/tazobactam. Interestingly, the clinical findings: Patterson J E et al., and Lee J et al. have found that increased use of piperacillin/tazobactam did not result in increased resistance, as in the case of other antibiotics. This could be due to the notion that several events may need to occur for resistance to be conveyed against piperacillin/tazobactam.

The study observed similarities in concordance for most antibiotics (11/14) between monomicrobial and polymicrobial specimens. However, three antibiotics, including vancomycin, meropenem, and piperacillin/tazobactam, were associated with significantly lower concordance rates for polymicrobial infections than monomicrobial infections. The discordance rate increased due to the higher bacteria resistance relative to ABR genes detected for vancomycin and meropenem. The discordance may be due to the interactions with other organisms in a polymicrobial sample. On the other hand, discordance for piperacillin/tazobactam in polymicrobial samples were due to increased detection of resistance genes. Previous research showed the odds of resistance decreased for piperacillin/tazobactam with each additional organism present. The increased discordance may be due to a need to overexpress AmpC, as discussed earlier, and perhaps overexpression of AmpC is reduced with the introduction of additional species to the mix. Regardless, these three antibiotics are relatively strong and often reserved for highly resistant bacterial infections.

P-AST could not generate susceptibility results on 372 samples that PCR identified as containing exclusively fastidious bacteria because they do not grow in culture. Therefore, the study could not create a concordance rate analysis for these patients. However, the presence or absence of ABR genes in these samples was analyzed by PCR, which revealed 15 ABR genes in these samples. Due to the same reason of fastidious growth, traditional urine culture, and isolates-based antimicrobial susceptibility tests would also not be clinically feasible. Therefore, ABR gene results provide clinically valuable information for patients with an exclusive fastidious bacterial infection.

While the study tested for a relatively large number of ABR genes, it could only detect the genes for which there are primers. Also, the study was able to detect the presence or absence and did not quantify the ABR genes. Therefore, the study could not evaluate the concentration of the ABR genes relative to the bacteria's bio-load and its impact on antimicrobial susceptibility. Future studies in planning include updated ABR gene testing panels and quantitative approaches.

The study with a large sample size from multiple institutions showed a 60% concordance rate between the presence or absence of ABR genes and the P-AST test result. In the remaining discordant, 40% of the cases, the reliance on the M-PCR ABR report without the phenotypic data may lead to inappropriate treatment. Therefore, it is essential to provide physicians with ABR gene results and antimicrobial susceptibility test results, especially the P-AST results, which take into consideration bacterial interactions, to assist them with clinical treatment decision-making.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Embodiments of the present invention can be freely combined with each other if they are not mutually exclusive.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for treating a polymicrobial infection in a patient in need thereof, wherein the patient either has a polymicrobial infection or is suspected of having polymicrobial infection, said method comprises:
a) obtaining or having obtained a sample from a source of the polymicrobial infection or suspected polymicrobial infection in the patient;
b) subjecting or having subjected a first portion of the sample to genetic identification testing, wherein genetic identification testing detects and identifies one or more organisms in the sample;
wherein if genetic identification testing detects one or more organisms in the sample then the patient has a polymicrobial infection;
c) subjecting or having subjected a second portion of the sample to genetic resistance marker testing, wherein genetic resistance marker testing is effective for detecting and identifying one or more resistance genes that confers resistance to one or more therapeutic agents;
d) subjecting or having subjected a third portion of the sample to pooled phenotypic antibiotic resistance testing, wherein pooled phenotypic antibiotic resistance testing either or both: (i) identifies one or more therapeutic agents to which the polymicrobial infection is resistant, and or (ii) identifies one or more therapeutic agents to which the polymicrobial infection is susceptible, wherein organisms in the polymicrobial infection in the third portion of the sample are not first isolated before the pooled phenotypic antibiotic resistance testing;
e) applying results from (b), (c), and (d) to a predetermined set of thresholds in a database that indicates therapeutic agents that are effective for treating polymicrobial infections, wherein applying results from (b), (c), and (d) identifies at least one therapeutic agent that is effective for treating the polymicrobial infection in the patient; and
f) administering at least one therapeutic agent identified in (e) to the patient, wherein the at least one therapeutic agent is effective for treating the polymicrobial infection.

2. The method of claim 1 further comprising compiling a data set that includes one or more data points selected from: (i) results of phenotypic antibiotic resistance testing, (ii) results of genetic identification testing, (iii) results of genetic resistance marker testing, (iv) therapeutic agents to which the polymicrobial infection is expected to have increased resistance, (v) therapeutic agents to which the polymicrobial infection is expected to have decreased resistance, (vi) suggested therapeutic agents, and (vii) formulation of suggested therapeutic agents.

3. The method of claim 1, wherein the sample comprises urine, blood, plasma, cerebrospinal fluid, saliva, sputum, pulmonary lavage, vaginal secretions, wound lavage, biopsy tissue, wound swab, rectal swab, nasal swab, tissue, fecal matter, sperm sample, semen sample, or prostate fluid.

4. The method of claim 1, wherein the sample comprises urine and the polymicrobial infection is a urinary tract infection.

5. The method of claim 1, wherein the genetic identification testing comprises PCR, fluorescence in situ hybridization (FISH), culture, mass spectrometry, electrochemical biosensing, flow cytometry, automated biochemical identification, or a combination thereof.

6. The method of claim 1, wherein genetic resistance marker testing comprises PCR or sequencing.

7. The method of claim 6, wherein the one or more resistance genes is ErmA+Erm B, TEM, CTX-M group 1, SHV, VEB, OXA-1, CTX-M group 2, CTX-M group 9, CTX-M group 8/25, PER-1, PER-2, GES, blaNDM-1, VIM, KPC, IMP-2 group, IMP-1 group, OXA-23, IMP-16, IMP-7, OXA-72, OXA-40, OXA-58, OXA-48, NDM, blaOXA-48, QnrA, QnrB, mecA, ampC, FOX, ACC, DHA, MOX/CMY, BIL/LAT/CMY, vanA1, vanA2, vanB, vanC1, or vanC2-C3-2.

8. The method of claim 1, wherein the genetic resistance marker testing comprises genetic antibiotic resistance testing.

9. The method of claim 8, wherein genetic antibiotic resistance testing comprises PCR or sequencing.

10. The method of claim 8, wherein the genetic antibiotic resistance testing is effective for detecting and identifying one or more antibiotic resistance genes that confers resistance to one or more antibiotics.

11. The method of claim 10, wherein the one or more antibiotic resistance genes is ErmA+Erm B, TEM, CTX-M group 1, SHV, VEB, OXA-1, CTX-M group 2, CTX-M group 9, CTX-M group 8/25, PER-1, PER-2, GES, blaNDM-1, VIM, KPC, IMP-2 group, IMP-1 group, OXA-23, IMP-16, IMP-7, OXA-72, OXA-40, OXA-58, OXA-48, NDM, blaOXA-48, QnrA, QnrB, mecA, ampC, FOX, ACC, DHA, MOX/CMY, BIL/LAT/CMY, vanA1, vanA2, vanB, vanC1, or vanC2-C3-2.

12. The method of claim 1, wherein the pooled phenotypic antibiotic resistance testing comprises introducing fractions of the third portion of the sample to one or more media samples, each media sample comprising a therapeutic agent, incubating the media samples with the fractions, and subsequently measuring viability of organisms in the media samples after incubation.

13. The method of claim 1, wherein organisms of the polymicrobial infection are one or a combination of: Acinetobacter baumannii, Actinotignum schaalii, Aerococcus urinae, Aerococcus urinae, Alloscardovia omnicolens, Candida albicans, Candida glabrata, Candida parapsilosis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhea, Pantoea agglomerans, Proteus mirabilis, Providencia stuartii, Pseudomonas aeruginosa, Serratia marcescens, Staphylococcus aureus, coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1 &2, Adenovirus, or CMV.

14. The method of claim 1, wherein the therapeutic agent is one or a combination of a: penicillin, tetracycline, cephalosporin, quinolone, lincomycin, macrolide, sulfonamide, glycopeptide antibiotic, aminoglycoside, carbapenem, ansamycin, annamycin, lipopeptide, Fosfomycin, monobactam, nitrofuran, oxazolidinone, amphotericin B, isavuconazole, itraconazole, micafungin, Posaconazole, voriconazole, cidofovir, vidarabine, foscarnet, acyclovir, or valacyclovir.
